(19) United States
(12) Patent Application Publication
(10) Pub. No.: US 2010/0292533 A1
(43) Pub. Date: Nov. 18, 2010

(54) Endoscopic Cutter with Reconfigurable Guides

(75) Inventors: Hideyuki Kasahara, Tokyo (JP); Ken Yamatani, Tokyo (JP); Randal J. Kadykowski, South Lyon, MI (US); Lyne M. Charron-Keller, Brighton, MI (US)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignees: TERUMO CARDIOVASCULAR SYSTEMS CORPORATION, Ann Arbor, MI (US); OLYMPUS MEDICAL SYSTEMS CORPORATION, Tokyo (JP)

(21) Appl. No.: 12/466,084

(22) Filed: May 14, 2009

(51) Int. Cl. A61B 1/00

(52) U.S. Cl. 600/104

(57) ABSTRACT

A bipolar cutter has a cutter main body for inserting into the interior of a body and a treating electrode that is positioned against an object of examination (such as a blood vessel or a bleeding site) to be electrically cut and cauterized. A guiding member guides the object of examination toward the treating electrode as it is displaced in the axial direction of the cutter main body. The guiding member is comprised of a pair of protruding members and a notched groove or gap that leads to the treating electrode while in a first configuration. A gap adjusting mechanism causes the guiding member or the treating electrode to be displaced in the axial direction to reduce or eliminate the length of the gap so that the treating electrode can better reach flat tissue surfaces for treatment.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

1. Field of the Invention

The present invention relates to a surgical operating device for treating objects of examination.

2. Description of the Related Art

Surgical operating devices having monopolar or bipolar living tissue cutting apparatus are employed to treat (for example, coagulate or cut) objects of examination (living tissue) such as a blood vessel or a bleeding site positioned on the wall (surface) of a body cavity.

For example, such a living tissue cutting apparatus is disclosed in Japanese Patent Publication No. 3839320. This living tissue cutting apparatus is comprised of a main body that is inserted into the patient's body; a treating tip member, provided on the tip of the main body for treating living tissue; and electrodes, provided on the treating tip member, for electrically treating living tissue. The treating tip member comprises a guiding member that guides the living tissue from the tip of the treating tip member to electrodes positioned on the base end of the treating tip member as the main body is main body is displaced.

For example, a blood vessel is guided to the electrodes by the guiding member. However, since the guiding member protrudes from the electrodes toward the tip side, there is a risk that, when treating a bleeding site located on a wall or flat surface within a body cavity, the bleeding site will not be guided to the electrodes due to the guiding member catching on the wall or pushing it away from the electrodes. That is, there is a risk that the electrodes of the living tissue cutting apparatus will not be pressed against the bleeding site because of the presence of the guiding member in that situation. Thus, there is a risk that this particular type of object of examination will not be treated, depending on where it is located. Further, when the object being treated is located on a wall or flat surface, such as in the case of a bleeding site within an internal body cavity being created as part of an endoscopic procedure, there is a risk that great effort may be required to press the electrodes against the wall when employing such a living tissue cutting apparatus.

### SUMMARY OF THE INVENTION

Hence, one object of the present invention, devised in light of the above problems, is to provide a surgical operating device that readily permits the treatment of an object of examination by facilitating the pressing of electrodes against flat surfaces of examination while retaining the ability to guide other non-flat tissue structures with a guide member.

In one aspect of the invention, an endoscopic surgical device comprises a main body for extending into a surgical cavity created within a patient's body, and a treating electrode deployable within the surgical cavity from a distal end of the main body to sever target tissue of the patient's body. A guiding member guides target tissue to the treating electrode as the treating electrode advances within the surgical cavity. The guiding member and the treating electrode are movable with respect to each other between a first configuration wherein the guiding member extends distally beyond the beyond the treating electrode and a second configuration wherein the extension of the guiding member distally beyond the treating electrode is less than the first configuration. The first configuration is adapted to sever target tissue comprised of narrow tissues in the patient's body. The second configuration is adapted to sever target tissue comprising substantially flat tissue surfaces of the surgical cavity.

Advantages of the invention will be set forth in the description which follows, and may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is an exploded lateral view of an endoscopic tissue harvesting device incorporating a surgical operating device (bipolar cutter) relating to Embodiment 1 of the present invention.

FIG. 2A is a perspective view of a trocar.

FIG. 2B is a lateral, longitudinal sectional view of a trocar.

FIG. 3 is a lateral, longitudinal sectional view of a treating sheath with the rigid endoscope removed.

FIG. 4 is a longitudinal sectional plan view of a treating sheath with the rigid endoscope removed.

FIG. 5 is a lateral, longitudinal sectional view of a treating sheath with the rigid endoscope inserted.

FIG. 6 is a longitudinal sectional plan view of a treating sheath with the rigid endoscope inserted.

FIG. 7 is a view from the direction of arrow A in FIG. 5.

FIG. 8 is a lateral, longitudinal sectional view of the tip member of a dissector.

FIG. 9A is a perspective view of a tissue harvesting device.

FIG. 9B is a schematic perspective view of the tip member.

FIG. 9C is a schematic front view of the tip member with portions of the protruding members omitted.

FIG. 10A is a view of a bipolar cutter from above.

FIG. 10B is a view of a bipolar cutter from below.

FIG. 11A is a view of a blood vessel holder from above.

FIG. 11B is a lateral, longitudinal sectional view of a blood vessel holder.

FIG. 11C is a front view of a blood vessel holder.

FIG. 12A is a view of a wiper from above.

FIG. 12B is a sectional view along section line B-B in FIG. 12A.

FIG. 13 is a perspective view of the wiper operating member.

FIG. 14 is a drawing of a skin incision in a lower limb.

FIG. 15 is a sectional view of a trocar installed in a lower limb skin incision, with a dissector inserted into a cavity guided by the trocar.

FIG. 16 is a comprehensive structural diagram showing a treating sheath inserted into a cavity guided by a trocar.

FIG. 17 is a drawing showing a monitor image.

FIG. 18 is a sectional view of a treating sheath inserted into a cavity.

FIG. 19 is a sectional view of the treatment state within a cavity.

FIG. 20 is a drawing showing a monitor image.

FIG. 21A is a perspective view of the operation of a blood vessel holder.

FIG. 21B is a perspective view of the operation of a blood vessel holder.

FIG. 21B is, FIG. 21C is a perspective view of the operation of a blood vessel holder.

FIG. 22 is a view of the treatment state within a cavity.

FIG. 23 is a drawing showing a monitor image.

FIG. 24 is a drawing showing a monitor image.

FIG. 25A is a plan view showing the operation of a bipolar cutter when the object of examination is a blood vessel, for example.

FIG. 25B is a plan view showing the operation of a bipolar cutter when the object of examination is a blood vessel, for example.

FIG. 25C is a plan view showing the operation of a bipolar cutter when the object of examination is a blood vessel, for example.

FIG. 25D is a plan view showing the operation of a bipolar cutter when the object of examination is a bleeding site, for example.

FIG. 25E is a plan view showing the operation of a bipolar cutter when the object of examination is a bleeding site, for example.

FIG. 25F is a plan view showing the operation of a bipolar cutter when the object of examination is a bleeding site, for example.

FIG. 26A is a sectional view of the interior of a cavity showing the operation of a bipolar cutter.

FIG. 26B is a sectional view of the interior of a cavity showing the operation of a bipolar cutter.

FIG. 27 is a section view of the interior of a cavity showing the treatment state.

FIG. 28 is a drawing showing a monitor image.

FIG. 29 is a perspective view of the tip member of a treating sheath.

FIG. 30 is a perspective view of the tip member of a treating sheath.

FIG. 31 is a perspective view of the tip member of a treating sheath.

FIG. 32A is a plan view showing the operation of a bipolar cutter with a protrusion gripped between protruding members when the object of examination is a bleeding site, for example.

FIG. 32B is a plan view showing the operation of a bipolar cutter with a protrusion gripped between protruding members when the object of examination is a bleeding site, for example.

FIG. 33A is a view from above of a bipolar cutter in a first variation example of Embodiment 1.

FIG. 33B is a drawing showing the operation of the bipolar cutter shown in FIG. 33A.

FIG. 33C is a drawing showing the operation of the bipolar cutter shown in FIG. 33A.

FIG. 33D is a drawing showing the operation of the bipolar cutter shown in FIG. 33A.

FIG. 33E is a drawing showing the operation of the bipolar cutter shown in FIG. 33A.

FIG. 34A is a view from above of a bipolar cutter when the protruding members are elastic members, for example.

FIG. 34B is a lateral view showing the operation of the bipolar cutter shown in 34A.

FIG. 34C is a lateral view showing the operation of the bipolar cutter shown in 34A.

FIG. 35A is a view from above of a bipolar cutter in a second variation example of Embodiment 1.

FIG. 35B is a plan view showing the operation of the bipolar cutter shown in 35A.

FIG. 35C is a view from above of a bipolar cutter when the bipolar cutter is in the form of a truncated cone.

FIG. 35D is a plan view showing the operation of the bipolar cutter shown in 35C.

FIG. 35E is a plan view showing the operation of the bipolar cutter shown in 35C.

FIG. 35F is a view from above of a bipolar cutter when the protruding members are integrated into a single member.

FIG. 35G is a plan view showing the operation of the bipolar cutter shown in FIG. 35F.

FIG. 36A is a view from above showing a bipolar cutter in an Embodiment 2.

FIG. 36B is a drawing showing the operation of the bipolar cutter shown in FIG. 36A.

FIG. 37A is a view from above of a bipolar cutter in a first variation example of Embodiment 2.

FIG. 37B is a drawing showing the operation of the bipolar cutter shown in FIG. 37A.

FIG. 38A is a view from above of a bipolar cutter in a second variation example of Embodiment 2.

FIG. 38B is a view from above of the bipolar cutter shown in FIG. 38A.

FIG. 39A is a view from above of a bipolar cutter in an Embodiment 3.

FIG. 39B is a lateral view of a bipolar cutter in Embodiment 3.

FIG. 39C is a drawing showing the operation of the bipolar cutter shown in 39A.

FIG. 39D is a drawing showing the operation of the bipolar cutter shown in 39B.

### DETAILED DESCRIPTION OF PREFERRED EMBODIMENTS

Embodiments of the present invention are described in detail below with reference to the drawings.

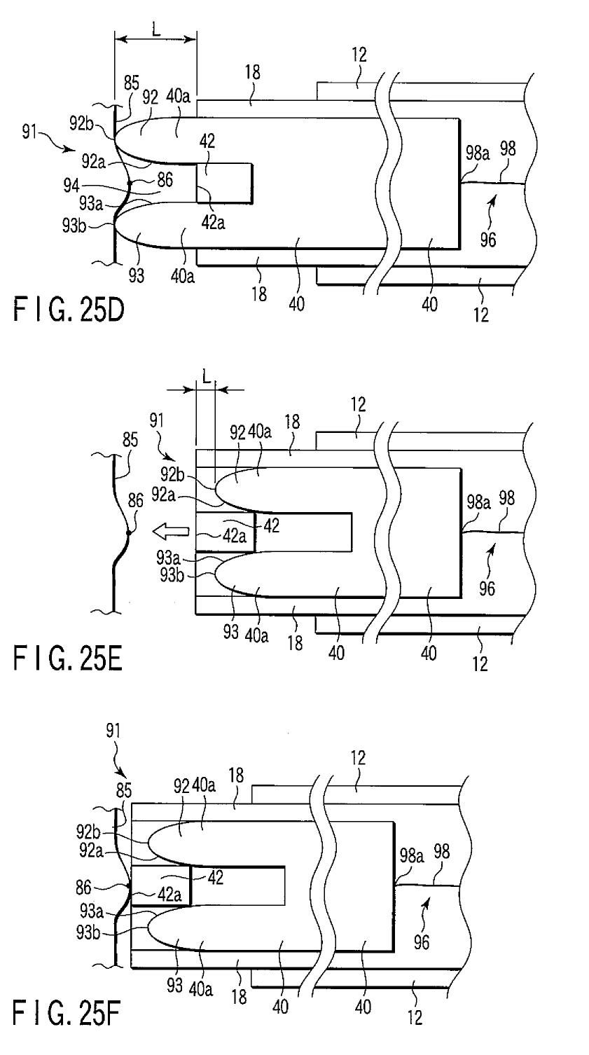

In the embodiments of the invention set forth below, the “object of examination” is a living tissue of a patient, such as a blood vessel 61 within a body cavity, a lateral branch 72 severed from a blood vessel 61 (FIGS. 15-24), or a bleeding site 86 positioned on a wall 85 or other substantially flat surface within a body cavity (FIGS. 25D-25F). The body cavity may be a natural cavity or may be one created for the purpose of an endoscopic procedure such as the harvesting of a blood vessel for blood vessel for cardiac bypass surgery. “Treating” includes incising, excising, boring a hole in, separating, coagulating, halting bleeding, collecting, cauterizing, and severing.

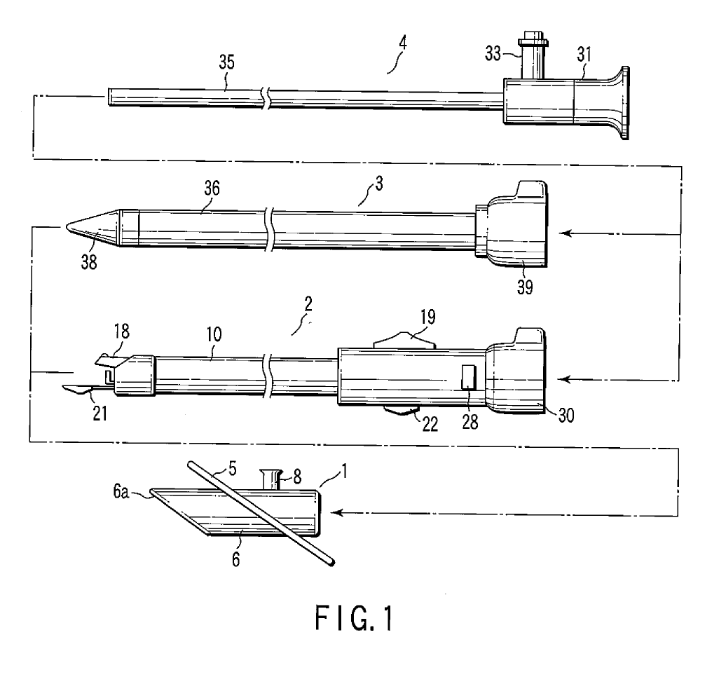

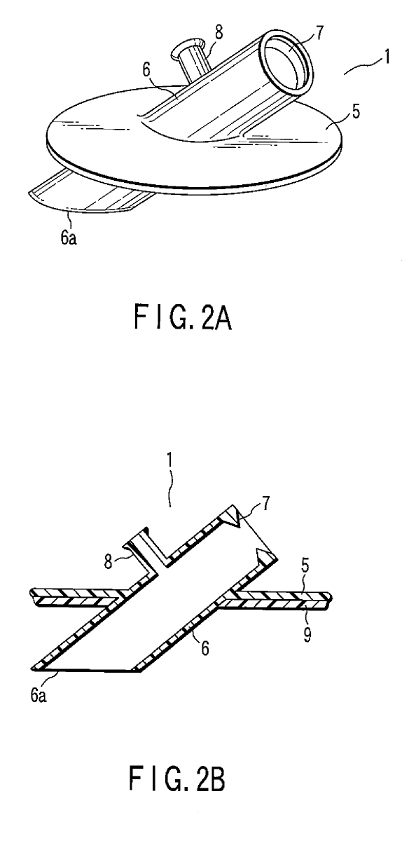

Embodiment 1 will be described with reference to FIGS. 1 to 5.

FIG. 1 shows an endoscopic tissue harvesting device, incorporating a surgical operation device relating to Embodiment 1 of the present invention, which device is comprised of a trocar 1, treating sheath 2, expansion means in the form of a dissector 3, and endoscope in the form of a rigid endoscope unit 4.

In trocar 1, an airtight ring 7 is provided on the inner circumferential surface in the base end portion of a guide tube 6.

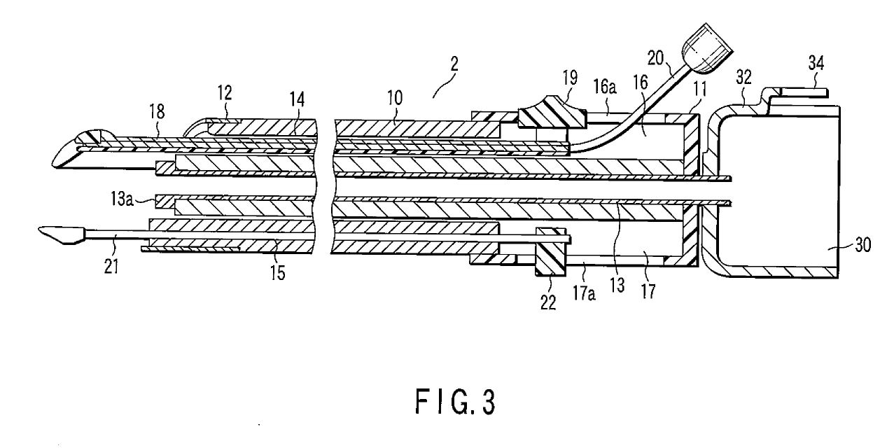

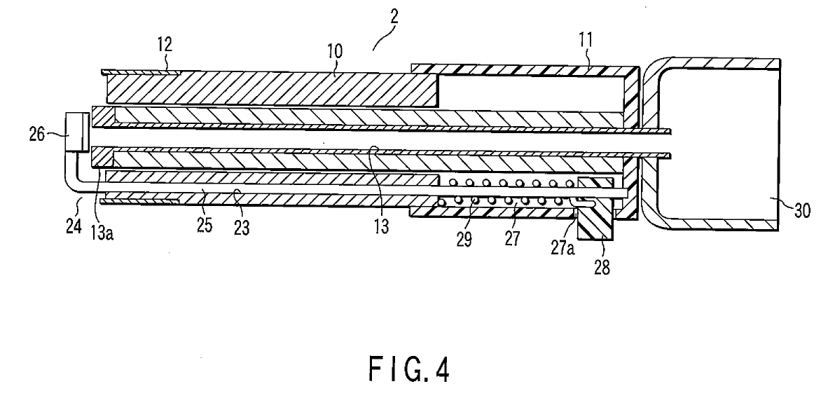

Treating sheath 2 will be described next with reference to FIGS. 3 and 4. Sheath main body 10 is of straight, cylindrical shape and is comprised of a synthetic resin material. A lubricating coating is provided on the outer surface of sheath main body 10 to permit ready sliding during insertion, for example. A cylindrical operating member cover 11, comprised of a grip member, is inset into the near end of sheath main body 10, and a tip cover 12 is inset into the far end.

A full-length endoscopic channel 13 is provided in the axial center portion of sheath main body 10. The near end of endoscopic channel 13 passes through operating member cover 11 and protrudes to the base side; and a flange member 13a, protruding from the front end surface of sheath main body 10, is provided on the far end thereof. Within sheath main body 10, a first treating apparatus channel 14 is provided at an off-center position on the upper side and a second treating apparatus channel 15 is provided at an off-center position on the lower side, on either side of endoscope channel 13. Accordingly, first treating apparatus channel 14 and second treating apparatus channel 15 are positioned symmetrically at positions as far removed as possible on either side of endoscope channel 13.

The near end of first treating apparatus channel 14 opens into first slide operation region 16 within operating member handle 11, and the near end of second treating apparatus channel 15 opens into second slide operating region 17 within operating member handle 11. A surgical operating device (high-frequency treating apparatus) in the form of a bipolar cutter 18, described further below, is inserted into first treating apparatus channel 14 in a manner permitting unencumbered advancement and retraction in an axial direction. A treating apparatus operating member 19, axially slidable over the range of a slot 16a in first slide operating region 16, is provided in the near end of first treating apparatus channel 14. A bipolar cable 20 is connected to bipolar cutter 18, and said bipolar cable 20 is led to the exterior through slot 16a.

A blood vessel holder 21, described further below, is inserted into second treating apparatus channel 15 in a manner permitting unencumbered advancement and retraction in an axial direction. A holder operating member 22, axially slidable over the range of a slot 17a in second slide operating region 17, is provided on the far end of second treating apparatus channel 15.

As shown in FIG. 4, a through-hole 23 is provided in an axial direction on one side of endoscope channel 13 within sheath main body 10. The wiper rod 25 of a wiper 24, described further below, is inserted circumferentially rotatably into through-hole 23.

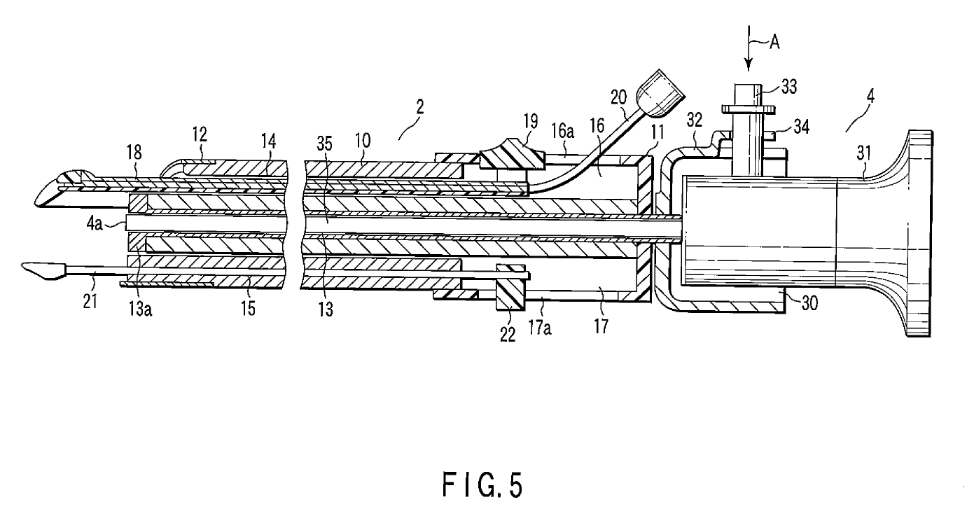

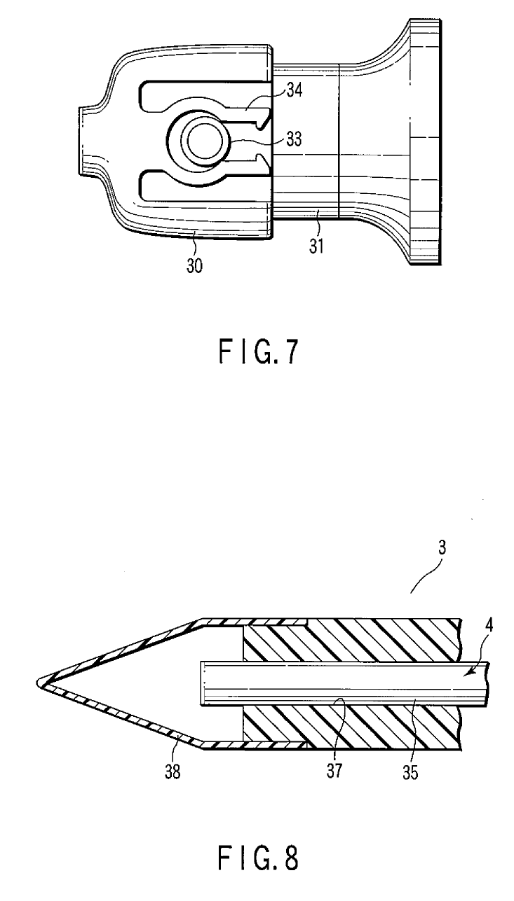

As shown in FIGS. 5, 6, and 7, an endoscope holding member 30 is provided in a fixed state in endoscope channel 13 on the near end side of operating member cover 11. Endoscope holding member 30 comprises an internal cavity adequate to contain the eyepiece 31 of rigid endoscope 4. A notched member 34 is provided in a portion (upper portion) of circumferential wall 32 of endoscope holding member 30. A light guide base 33 provided on eyepiece member 31 is inserted into and engages with notched member 34.

Accordingly, rigid endoscope 4 is supported by treating sheath 2 and axially positioned when insertion member 35 of rigid endoscope 4 is inserted into endoscope channel 13, light guide base 33 is inserted into and engages with notched member 34, and endoscope holding member 30 is supported by eyepiece member 31. Sheath main body 10 and operating member cover 11 are rotatably fixed in endoscope channel 13. Endoscope channel 13 and endoscope holding member 30 are secured. Thus, when treating sheath 2 and rigid endoscope 4 have been assembled, the tip beyond tip cover 12 is rotatably held with respect to rigid endoscope 4.

Dissector 3 will be described next with reference to FIGS. 1 and 8. Insertion cylinder member 36 is of straight, cylindrical shape. An insertion passage 37, through which the insertion member 35 of rigid endoscope 4 is inserted, is provided in the axial center portion of insertion cylinder member 36. A lubricating coating is provided on the outer surface of insertion cylinder member 36 to facilitate sliding during insertion. A separating member 38, in the form of a conical cylinder, is secured by a transparent synthetic resin material on the far end of insertion cylinder member 36. An endoscope holding member 39 is provided on the near end of insertion cylinder member 36. Endoscope holding member 39 holds eyepiece member 31 of rigid endoscope 4. Endoscope holding member 39 is desirably configured identically with endoscope holding member 30 of treating sheath 2.

Bipolar cutter 18, which is a surgical operating device relating to Embodiment 1 of the present invention, will be described next.

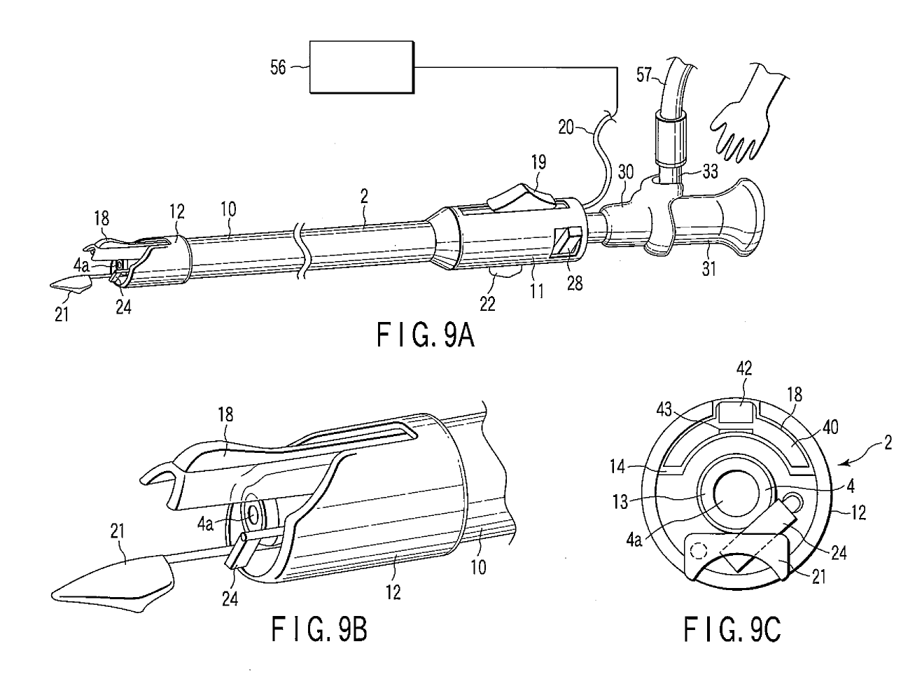

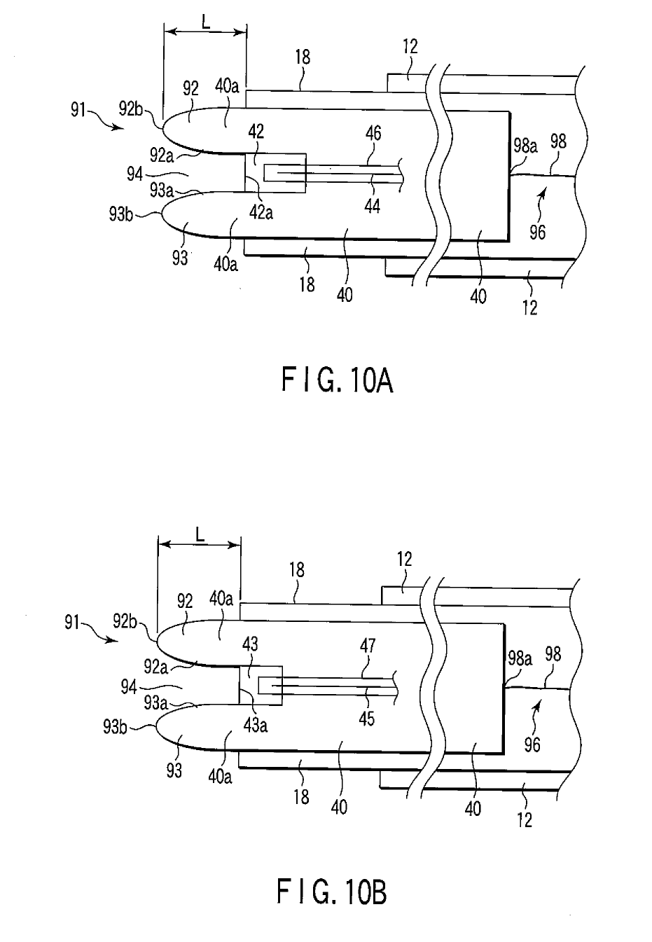

As shown in FIGS. 9A, 9B, 9C, 10A, and 10B, bipolar cutter 18 is comprised of a cutter main body 40 that is inserted into the interior of a patient's body; a treating tip member 40a, positioned on the tip of cutter main body 40, for treating (e.g., coagulating and/or cutting) an object of examination; and treating main body members in the form of electrodes 42 and 43, positioned on treating tip member 40a, for electrically treating an object of examination.

Cutter main body 40 is comprised of an insulating material (e.g., a ceramic), such as a synthetic resin material.

Cutter main body 40 is covered by a roof member in the form of a belt-like plate of arcuate, curved sectional shape, so as to run along the inner circumferential, arcuate surface of sheath main body 10. The roof member, as described further below, serves to ensure the field of view of rigid endoscope 4 by preventing downward sagging of tissue from above (pushing away fatty tissue within body cavities).

Further, a guiding member 91, guiding an object of examination such as a blood vessel 61 or a bleeding site 86 toward electrodes 42 and 43 as cutter main body 40 moves axially, is provided around electrodes 42 and 43 within treating tip member 40a. In the present embodiment, guiding member 91 is comprised of a pair of parallel protruding members 92 and 93 that define a notched groove or gap 94 between them.

Protruding members 92 and 93 are tip-tapered in shape, with the base progressively widening relative to the tip. As cutter main body 40 moves, protruding members 92 and 93 guide target tissue (e.g., narrow tissue such as a blood vessel) from the tip distal ends 92b and 93b toward electrodes 42 and 43 positioned on the base end of protruding members 92 and 93 at the proximal edge of gap 94.

Gap 94 is formed by the side edge 92a of protruding member 92 facing protruding member 93 and the side edge 93a of protruding member 93 facing protruding member 92. There is no particular limitation on the shape of protruding members 92 and 93; they need only run axially along cutter main body 40. Gap 94 can also be a V-groove that is cut in the shape of a “V,” for example.

Protruding members 92 and 93 are symmetrically positioned in a direction orthogonal to the distal edges 42a and 43a of electrodes 42 and 43 on cutter main body 40. Protruding members 92 and 93, together with cutter main body 40, are made to slide axially along bipolar cutter 18 (e.g. extending through tip cover 12 of sheath main body 10) by a gap adjusting mechanism 96, described further below. That is, protruding members 92 and 93 are caused to protrude from within bipolar cutter 18 by gap adjusting mechanism 96, or are contained within bipolar cutter 18. Protruding members 92 and 93 are formed of thin, sheet-like metal, such as nitinol and other shape memory alloys.

Guiding member 91 comprises protruding members 92 and 93 and gap 94, but need not be limited thereto so long as it is capable of guiding the object of examination to electrodes 42 and 43, and suffices to comprise a groove, formed by two sides, for guiding the object of examination to electrodes 42 and 43.

In the present embodiment, cutter main body 40 includes a gap adjusting mechanism 96 for adjusting as desired the length L of gap 94 between electrodes 42 and 43 and distal edges 92b and 93b of guiding member 91 in the longitudinal axial direction of cutter main body 40 in conformity to the shape of the object of examination, such as a blood vessel 61 or a bleeding site 86, as guiding member 91 guides the object of examination toward electrodes 42 and 43. More specifically, gap adjusting mechanism 96 causes guiding member 91 to be displaced in the axial direction of tip cover 12 to adjust as desired length L between the tip of guiding member 91 (tip members 92b and 93b of protruding members 92 and 93) and the tips 42a and 43a of electrodes 42 and 43 between a maximum (providing gap 94 with its maximum depth) and a minimum (providing gap 94 with a minimum depth). The minimum depth may correspond to a zero depth (i.e., electrode tips 42a and 43a are at the same position or extend distally beyond distal ends 92b and 93b of guiding member 91), for example. In its normal or first configuration, guiding member 91 protrudes from tips 42a and 43a with length L at its maximum, but gap adjusting mechanism 96 adjusts length L to a second configuration wherein the extension of guiding member 91 distally beyond electrodes 42 and 43 is less than in the first configuration.

Gap adjusting mechanism 96 preferably includes the above-described cutter operating member 19 on handle 11 and an operating wire 98. FIGS. 10A and 10B show tip 98a of operating wire 98 connected to cutter main body 40. The base end of wire 98 (not shown) is connected to cutter operating member 19 (e.g., a thumb lever) to adjust length L as desired by pushing and pulling protruding members 92 and 93 by means of operating member 19, thereby displacing protruding members 92 and 93 in the longitudinal direction of cutter main body 40. The operation of cutter operating member 19 and the pulling of operating wire 98 by means of cutter operating member 19 causes protruding members 92 and 93 and cutter main body 40 to move in the axial direction of bipolar cutter 18, thereby adjusting length L.

In this process, for example, guiding member 91 can be retracted (moved) to be roughly even with electrodes 42 and 43, or further to the base end side of bipolar cutter 18 than electrodes 42 and 43, and contained within bipolar cutter 18. Thus, electrodes 42 and 43 are roughly even with guiding member 91, or are positioned more to the tip side than guiding member 91, being exposed on guiding member 91.

A pair of opposed electrodes 42 and 43 are secured to bipolar cutter 18 and positioned on the bottom of gap 94. Electrodes 42 and 43 are treating main body members (cutting members) that electrically treat (for example, cut and cauterize) the object of examination. Pair of electrodes 42 and 43 are not positioned within the same plane, but are positioned opposite each other above and below bipolar cutter 18. When gap 94 is a “V” groove, pair of electrodes 42 and 43 are positioned at the intersection of the sides (such as sides 92a and 92b) having the same V-shape.

Of the two electrodes, the outer surface area of upper electrode 42 is greater than that of lower electrode 43. That is, the surface area of electrode 42 coming into contact with tissue is large, and the surface area of lower electrode 43 coming into contact with tissue is small. Thus, lower electrode 43 is made to function as a cutting (severing) electrode, and upper electrode 42 is made to function as a coagulating electrode.

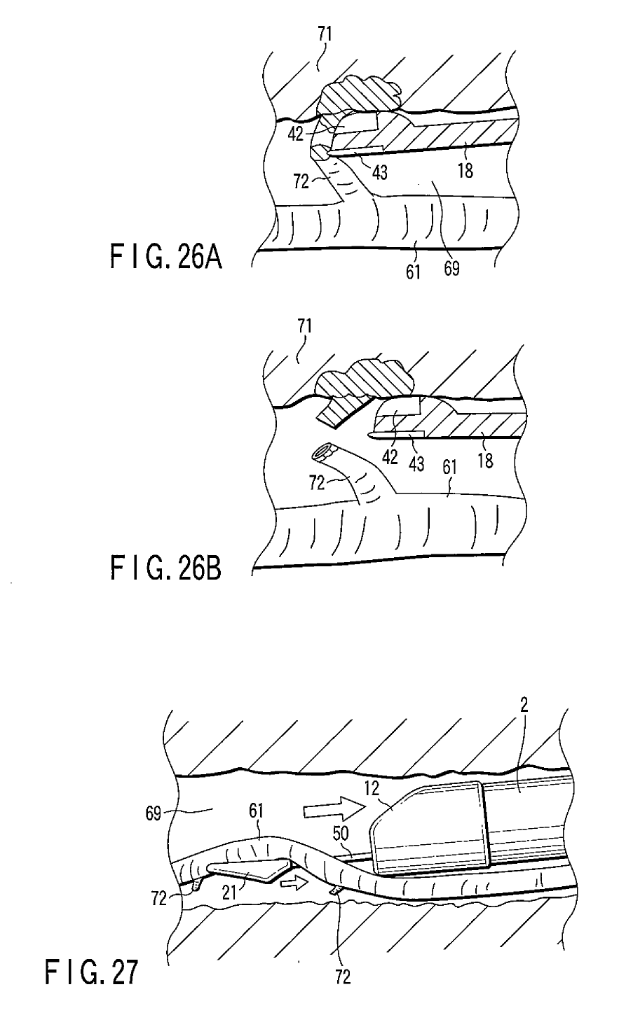

Generally, an electrode with a large contact surface area is better able to stop bleeding during cutting than an electrode with a small contact surface area. As set forth further below (see FIGS. 26A, 26B, 27, and the like), the site of the cut at an incised side branch 72 of a blood vessel 61 that is being isolated is ligated with sutures once blood vessel 61 has been isolated. However, since the site of the cut in the patient remains as is within the patient, bleeding is desirably stopped. Thus, in the present embodiment, electrode 43, with a small contact surface area operating as a cutting electrode, is positioned beneath, that is, on the side of blood vessel 61 which is to be isolated (the side of blood vessel holder 21, described further below, that holds blood vessel 61). Electrode 42, with a large contact surface area operating as a coagulating electrode, is positioned above, that is, on the side remaining in the body. Electrode 42 with a large contact surface area is positioned above, on the body side (the side remaining in the body), to minimize the thermal effects on blood vessel 61 by separating electrode 42 as far as possible from the blood vessel 61 being collected. Accordingly, upper electrode Accordingly, upper electrode 42 will be referred to below as the body-side electrode, and lower electrode 43 as the cut electrode.

Lead wires 44 and 45 are connected to body-side electrode 42 and cut electrode 43, respectively. Lead wires 44 and 45 run along the upper surface and lower surface of cutter main body 40, connecting to bipolar cable 20. Further, lead wires 44 and 45 are clad with and insulated by insulating films 46 and 47. Portions of bipolar cutter 18 other than electrodes 42 and 43 can also be formed of a transparent material (such as polycarbonate).

Blood vessel holder 21 will be described next with reference to FIGS. 11A, 11B, and 11C. Blood vessel holder 21 is formed of a synthetic resin material or the like that is roughly triangular in shape when viewed from above. The upper surface of blood vessel holder 21 is formed as a smooth surface 48. The lower surface of blood vessel holder 21 is formed as a circular arcuate concave surface 49. An operating rod 50 is linked at an off-center position on one side at the rear end of blood vessel holder 21. Operating rod 50 is inserted in freely advancing and retracting fashion into second treating apparatus channel 15.

A separating member 51 that separates tissue is formed on the tip of blood vessel holder 21. Separating member 51 has an acute angle. First taper surfaces 52a and 52b are formed bilaterally symmetrically on separating element 51. Inclined surfaces 53a and 53b, narrowing toward the tip, are formed on the upper and lower surfaces of separating member 51. The base portion of first taper surface 52a on the opposite side from the joint with operating rod 50 of blood vessel holder 21 is formed on a second taper surface 54 that is circular arcuate in form. Second taper surface 54 is connected to a hook member 55. Hook member 55 hooks blood vessel 61 at the rear end, comprising a smooth surface, of blood vessel holder 21.

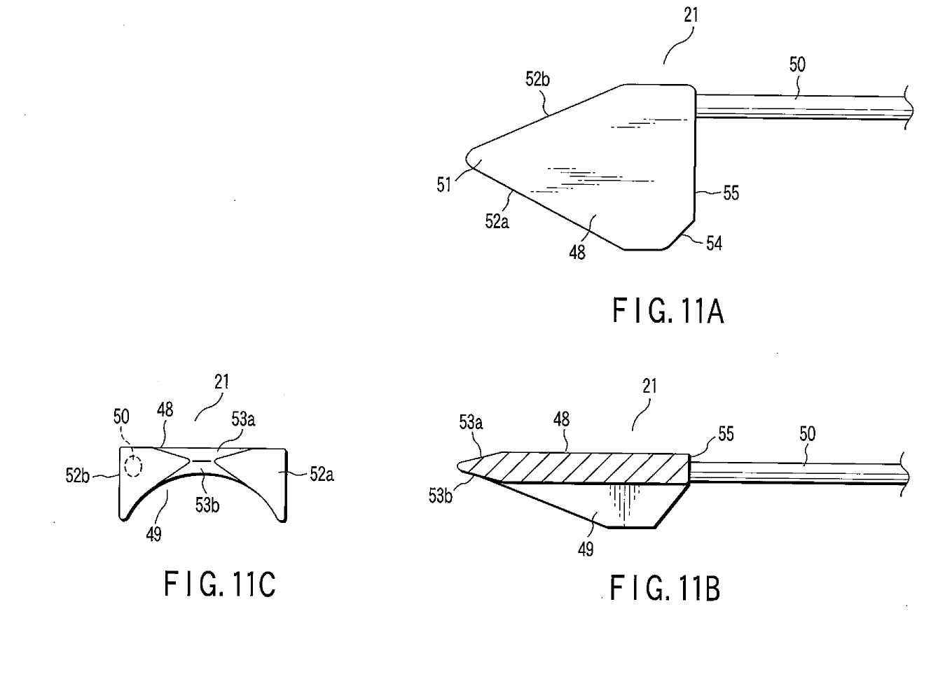

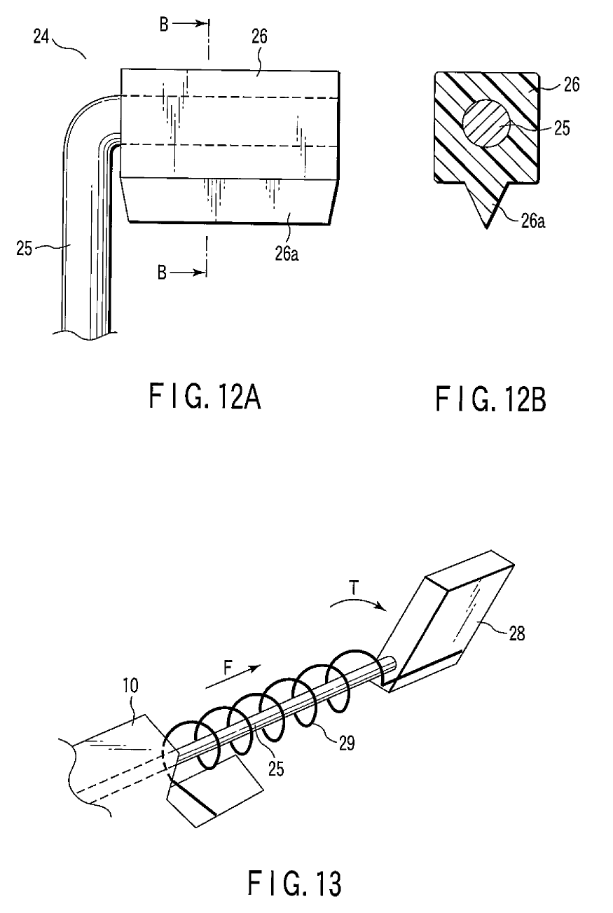

Wiper 24 will be described next with reference to FIGS. 12A and 12B. Wiper rubber 26 is secured to the far end of wiper rod 25. Wiper rubber 26 is secured by adhesion, insert molding, or the like to the bend in the “L”-shape of the wiper rod, perpendicular to the axial direction of wiper rod 25. Wiper rubber 26 has a scraping member 26a. Scraping member 26a is triangular in cross-sectional shape and is flexible. Foreign matter such as blood, mucous, and fat that has adhered to object lens surface 4a of rigid endoscope 4 is scraped off by rotating wiper rubber 26. In this process, since scraping member 26a is flexible, any difference in level produced between the tip surface of sheath main body 10 and object lens surface 4a is overcome by scraping member 26a, which rubs against object lens surface 4a.

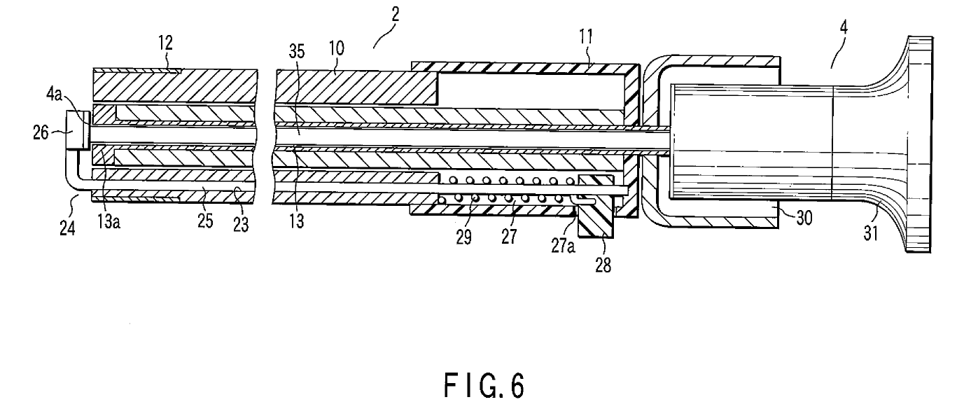

The near end of wiper rod 25, as shown in FIGS. 6 and 13, extends to the rotating operating member 27 within operating member cover 11, and is rotatably supported by the inner wall of operating member cover 11. A wiper operating member 28 is secured on the near end of wiper rod 25. Wiper operating member 28 is rotatable within the range of slot 27a in the circumferential direction of operating member cover 11.

A torsion coil spring 29 is provided within rotating operating member 27. As shown in FIG. 13, torsion coil spring 29 is comprised of a coil spring provided in wiper rod 25 of wiper 24. As shown in FIGS. 6 and 13, one end of torsion coil spring 29 is in contact with the end surface of sheath main body 10, and the other end of torsion coil spring 29 is in contact with the lateral surface of wiper operating member 28. Torsion coil spring 29 is inserted in a compressed state between the end surface of sheath main body 10 and wiper operating member 28. Thus, torsion coil spring 29 exerts a force on wiper 24 (wiper rubber 26) in the direction of the near end of sheath main body 10.

Torsion coil spring 29 is also secured between the end surface of sheath main body 10 and the lateral surface of wiper operating member 28. Thus, torsion coil spring 29 exerts a force on wiper rubber 26, causing it to retract toward objective lens surface 4a.

Thus, when there is a torque T rotating wiper rod 25 in the circumferential direction of wiper rod 25, torsion coil spring 29 generates a force F pushing wiper rod 25 toward the near end of sheath main body 10. Thus, wiper rubber 26 is pushed in a direction causing it to retract toward objective lens surface 4a, coming into contact with with objective lens surface 4a.

FIG. 6 shows insertion member 35 installed in endoscope channel 13. FIGS. 9A and 9B show bipolar cutter 18 and blood vessel holder 21 protrude from the tip (tip cover 12) of treating sheath 2. Bipolar cable 20 connects to high-frequency generating device 56, and light guide base 33 connects to light guide cable 57.

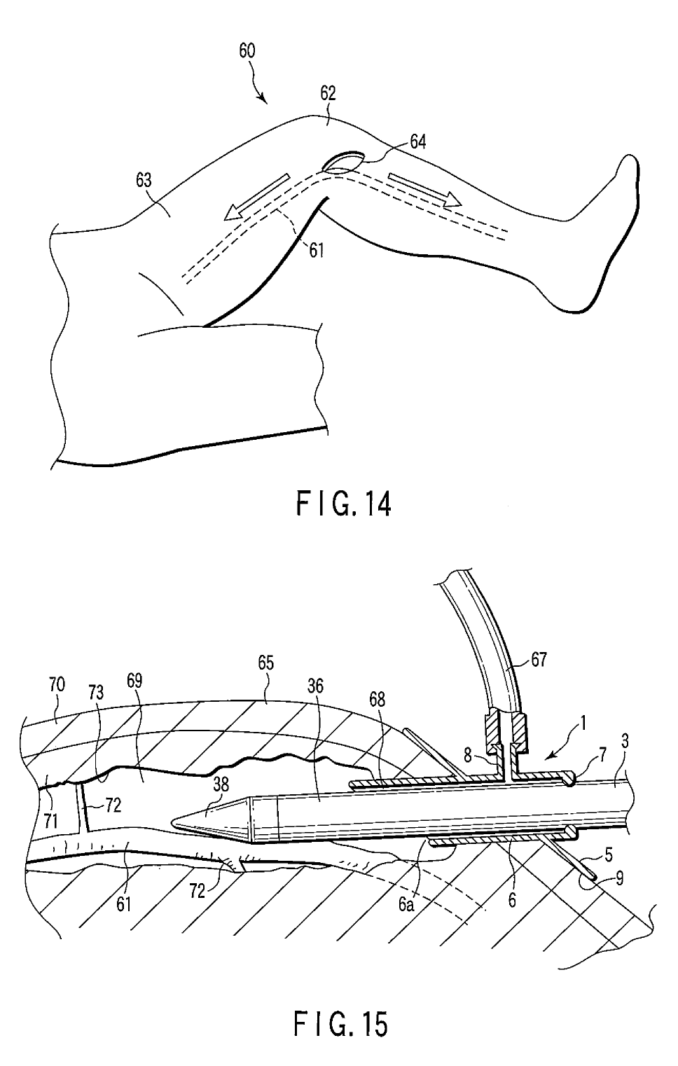

The treatment of an object of examination with a tissue harvesting device constituted as set forth above will be described next with reference to FIG. 14. An “object of examination” is, for example, a section of a blood vessel to be collected (namely, blood vessel 61), such as the great saphenous vein (FIG. 14) running from the groin region of the lower limbs 60 to the ankle, or a bleeding site 86 in a wall 85 within a body cavity (FIGS. 25D-25F).

First, in the course of collecting blood vessel 86 between the knee 62 and the inguinal region 63, an incision 64 is made directly above blood vessel 61 with a scalpel or the like at a site in the knee area.

Next, blood vessel 61 is exposed with a dissector or the like, not shown, in incision 64. The tissue directly above blood vessel 61 is then peeled back from incision 64 with a dissector or the like to a distance permitting observation with the naked eye.

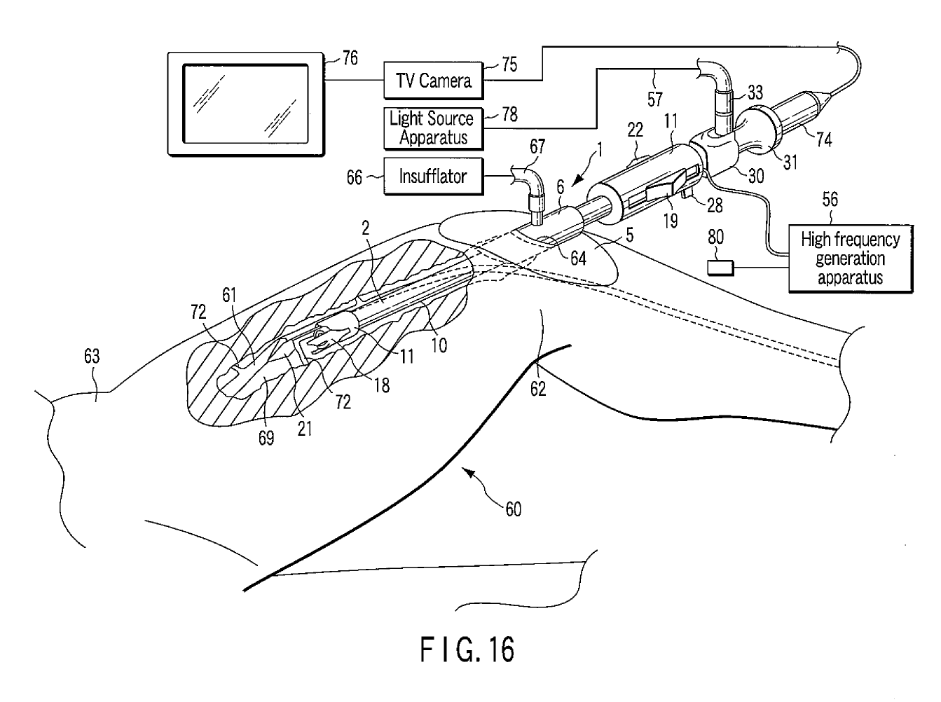

Next, as shown in FIGS. 15 and 16, progress of the ongoing dissection around blood vessel 61 by the dissector tip is picked up by means of a video camera 75 through a video camera head 74 connected to eyepiece member 31 and displayed on monitor 76 as a monitor image. In the course of inserting dissector tip 38 and running it along blood vessel 61, guide tube 6 is inserted at an angle (roughly parallel to blood vessel 61) toward the inguinal region 63. Once tip member 6a is facing downward, adhesive layer 9 is adhered and secured to the epidermis. In this state, an insufflation tube 67 that is connected to an insufflator 66 is connected to an insufflation port 8.

In this case, the outer circumferential surface of insertion cylinder member 36 is tightly secured to an airtight ring 7. Thus, guide tube 6 and cavity interior 69 are in an airtight state, and a gas introduction passage 68 is ensured between guide tube 6 and insertion cylinder member 36.

Further, light guide base 33 is connected to light source device 78 through a light guide cable 57. Accordingly, a light from the tip member of rigid endoscope 4, illuminates cavity interior 69. When insufflator 66 is driven, it sends gas (such as CO₂) into cavity interior 69 via insufflation tube 67, insufflation port 8, and gas introduction passage 68, causing cavity interior 69 to expand. The gas does not leak to the exterior at this time, since insertion cylinder member 36 is tightly attached to airtight ring 7. Accordingly, cavity interior 69 expands reliably.

Subcutaneous tissue is present in the lower layer of epidermis 65 within cavity interior 69, and blood vessel 61 is present within and beneath blood vessel connective tissue 71. One end of multiple side branches 72 are connected to blood vessel 61. Side branches 72 are part of forks in blood vessel 61. The other end of side branches 72 are connected to blood vessel connective tissue 71. Further, subcutaneous fat 73 is attached to blood vessel connective tissue 71.

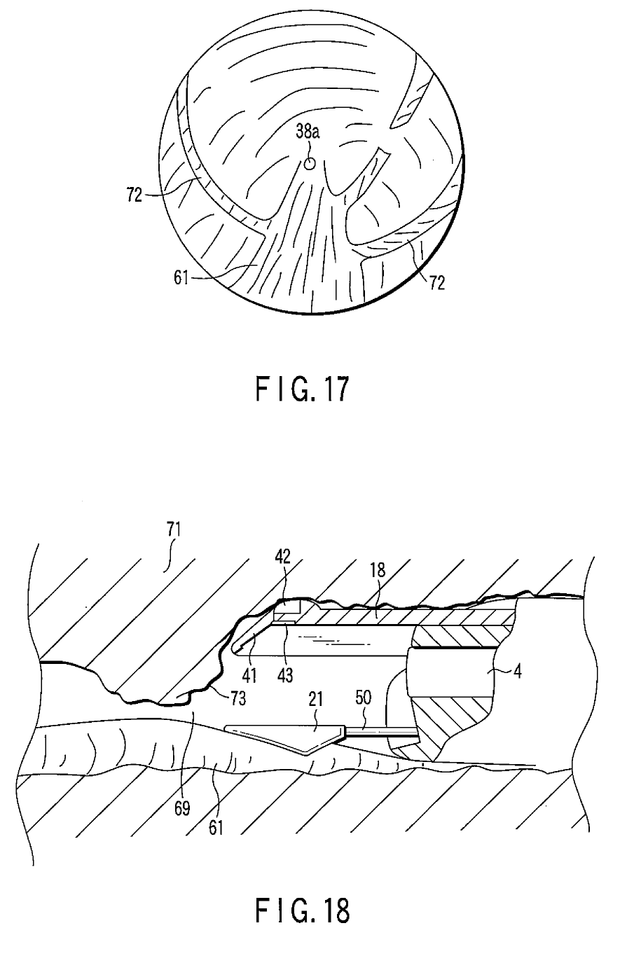

Checking the monitor image reveals a display such as that shown in FIG. 17. Monitor 76 displays blood vessel 61 and side branches 72. In FIG. 17, 38a is an image of the dissector tip 38.

Hence, when inserting dissector 3, gradual progress is made by means of an operation wherein said dissector is pushed in slightly as blood vessel connective tissue 71, blood vessel 61, and side branches 72 are separated by dissector tip 38 without damaging blood vessel 61 and side branches 72, and then slightly pulled back while observing cavity interior 69 on monitor 76. At this time, trocar 1 does not separate from epidermis 65 even in the event of horizontal or vertical movement of dissector 3, since trocar 1 is secured by adhesive layer 9 to epidermis 65. Dissector 3 thus passes from knee 62 to inguinal region 63 along blood vessel 61.

Once the task of separation with dissector 3 has been completed, dissector 3 is removed from trocar 1 and then rigid endoscope 4 is removed from dissector 3 with all cables still attached. As shown in FIG. 16, rigid endoscope 4 is next inserted into treating sheath 2. Treating sheath 2 is then inserted into guide tube 6.

When operating member cover (i.e., handle) 11 is grasped in one hand by the surgeon, for example, and holder operating member 22 is advanced with the thumb, for example, blood vessel holder 21 protrudes from tip cover 12. Further, when cutter operating member 19 is advanced with the index finger of the hand holding operating member cover 11, for example, bipolar cutter 18 protrudes from tip cover 12. That is, blood vessel holder 21 and bipolar cutter 18 are advanced and retracted while the surgeon is holding operating member cover 11 with one hand.

Accordingly, as shown in FIG. 18, when a large amount of subcutaneous fat 73 is present in the blood vessel connective tissue within cavity interior 69, and treating sheath 2 is pushed forward with bipolar cutter 18 protruding, cavity interior 69 is widened. At this time, bipolar cutter 18, due to the curved shape (roof shape) of cutter main body 40, prevents sagging of tissue from above (pushes away fatty tissue that is present within the body cavity), ensuring a good field of view for rigid endoscope 4. At this time, since the lower surface of blood vessel holder 21 is in the form of a circular arcuate concave surface 49, it can be advanced by sliding over the upper surface of blood vessel 61 without damaging blood vessel 61.

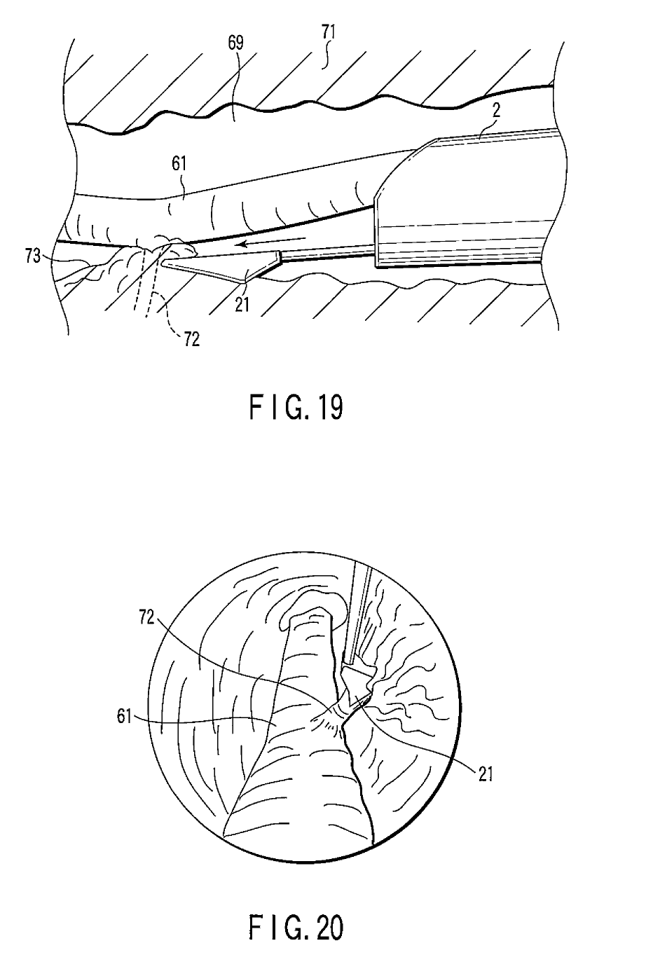

Further, as shown in FIG. 19, there are cases where side branches 72 are buried in subcutaneous fat 73. In such a case, blood vessel holder 21 protrudes from treating sheath 2 and penetrates subcutaneous fat 73, separating subcutaneous fat 73 from blood vessel 61. Rotating treating sheath 2 in its entirety in a circumferential direction within guide tube 6 rotates blood vessel holder 21, causing blood vessel holder 21 to separate subcutaneous fat 73 from side branches 72. The course of events at this time is displayed as a monitor image on monitor 76, as shown in FIG. 20. In this manner, monitor 76 allows the surgeon to determine the position of blood vessel holder 21 based on the monitor image, and prevents the surgeon from damaging blood vessel 61 or side branches 72.

When treating sheath 2 is pressed into cavity interior 69 as subcutaneous fat 73 is eliminated, holder 21 of the targeted blood vessel approaches a side branch 72. At this time, circular arcuate concave surface 49 comes into contact with the upper surface surface of blood vessel 61, advancing as it slides along the upper surface of blood vessel 61. Thus, damage to blood vessel 61 is prevented.

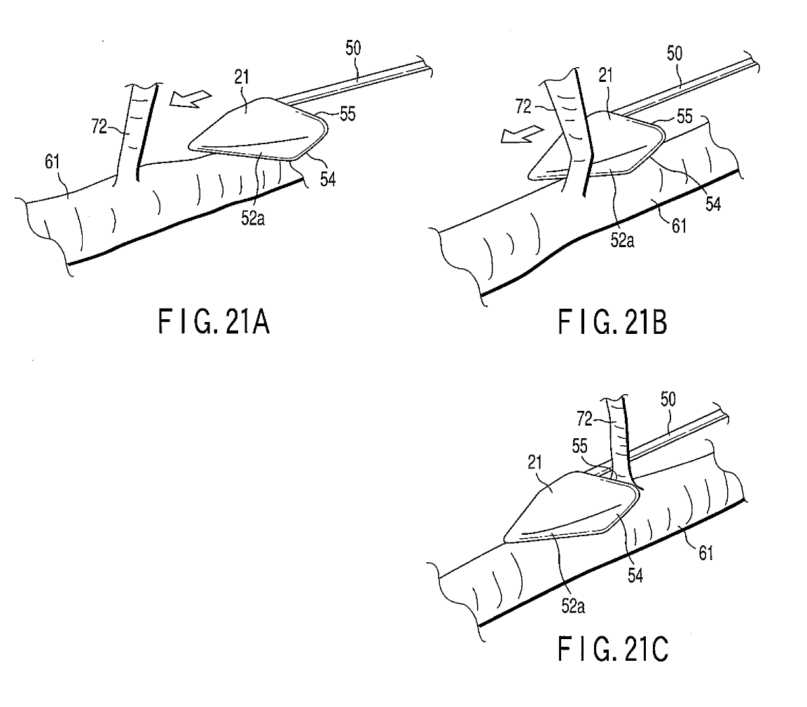

Further, FIGS. 21A, 21B, and 21C show the technique of holding a side branch 72 with blood vessel holder 21. Blood vessel holder 21 has a first taper surface 52a, with a second taper surface 54 formed in succession with first taper surface 52a. Thus, as blood vessel holder 21 advances, side branch 72 first comes into contact with taper surface 52a (see FIG. 21B).

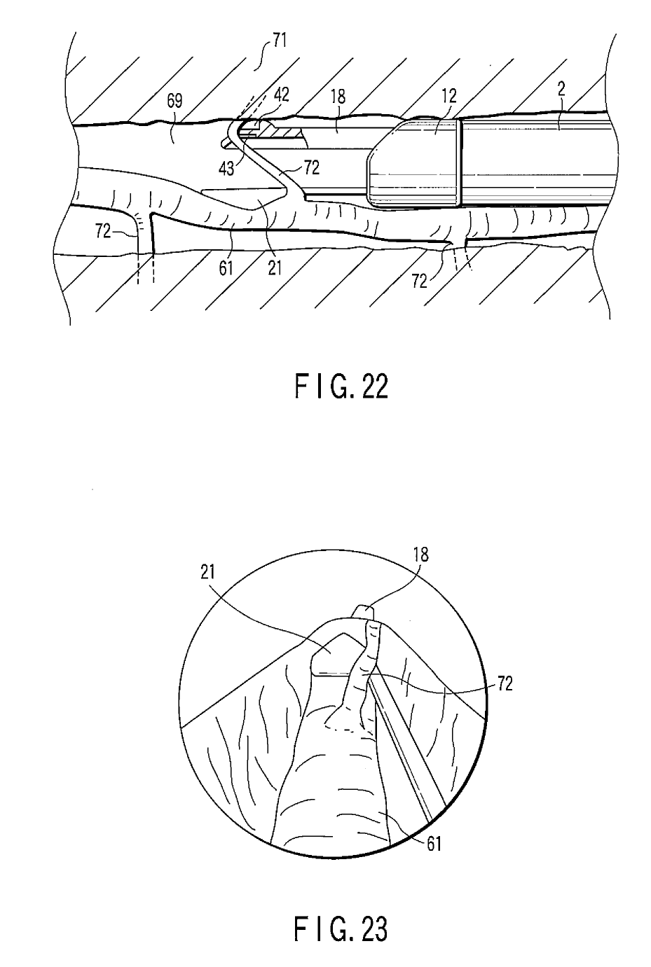

When blood vessel holder 21 advances, side branch 72 comes into contact with second taper surface 54 after first taper surface 52a. Subsequently, side branch 72 slides down to hook member 55 and is hooked (see FIG. 21C). In this manner, side branch 72 is readily held through the advancement operation of blood vessel holder 21. When blood vessel holder 21 is pulled to the front in this state, tension is applied to side branch 72, as shown in FIG. 22. FIG. 23 is a monitor image showing side branch 72 hooked by hook member 55. The monitor image allows the surgeon to confirm that side branch 72 is hooked on hook member 55. In this manner, when blood vessel holder 21 holds a side branch 72 not to the fore, but on the far side, side branch 72 will be positioned in front of the observation visual field and the area around side branch 72 is clearly confirmed by rigid endoscope 4. When a blood vessel holder 21 is present in front of a side branch 72, the observation visual field to the front is inhibited by blood vessel holder 21, creating the risk of improper determination of the positions of side branch 72 and blood vessel 61. Accordingly, as set forth further below, side branch 72 is safely treated (cut) without damaging blood vessel 61.

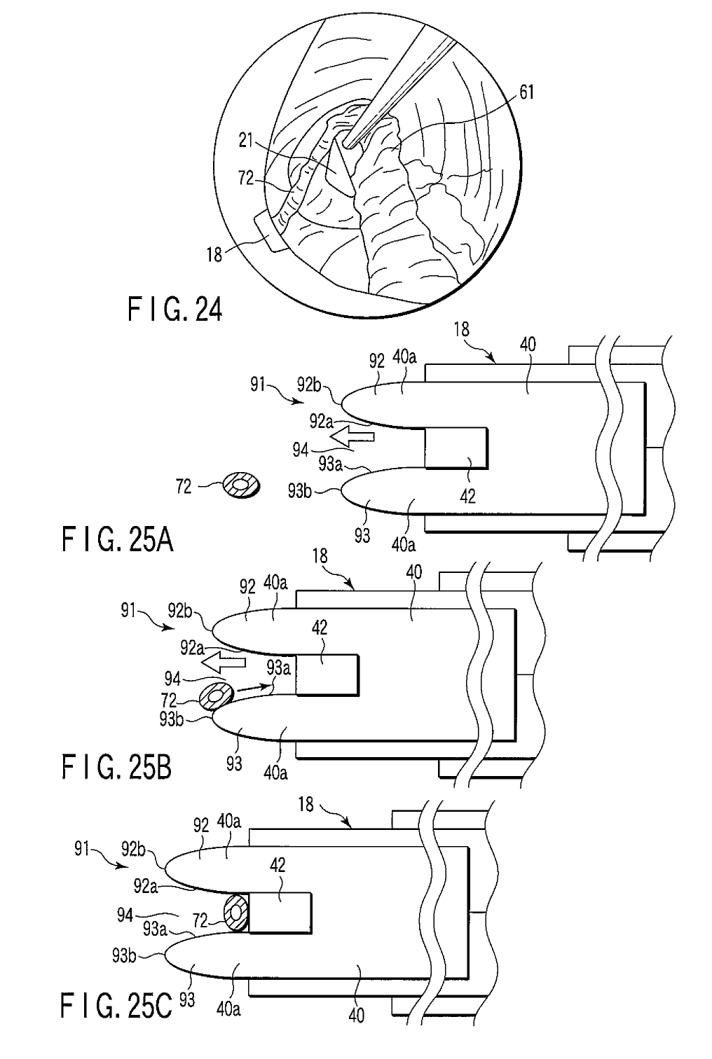

When the state shown in FIG. 23 occurs, the surgeon advances bipolar cutter 18 so that it approaches the side branch 72 being held by blood vessel holder 21. At this time, as shown in the monitor image of FIG. 24, blood vessel holder 21 retracts blood vessel 61 away from bipolar cutter 18 in such a manner that bipolar cutter 18 does not come into contact with blood vessel 61.

FIGS. 25A, 25B, and 25C show a technique for cutting a side branch 72 with bipolar cutter 18. Portions of the figures, such as lead wire 44 and insulation coating 46, have been omitted in FIGS. 25A, 25B, and 25C. When bipolar cutter 18 advances toward side branch 72, side branch 72 is guided to the electrodes 42 and 43 by edges 92a and 93a of gap 94. Accordingly, as shown in FIG. 26A, cutter electrode 43 comes into contact with side branch 72 and body side electrode 42 comes into contact with blood vessel connective tissue 71 or side branch 72. The surgeon energizes the electrodes with a high frequency current, resulting in separation and coagulation of side branch 72.

FIGS. 25D, 25E, and 25F show a technique for treating (cutting) a bleeding site 86 with bipolar cutter 18. Portions of the figures, such as lead wire 44 and insulation coating 46, have been omitted in FIGS. 25D, 25E, and 25F.

As shown in FIG. 25D, when the object of examination is a bleeding site positioned within a wall 85 positioned within a body cavity and bipolar cutter 18 is advanced toward wall 85 with protruding members 92 and 93 in the first configuration, they come into contact with wall 85 as shown in FIG. 25D. Before energizing electrodes 42 and 43, the cutter operating member (thumb lever) 19 is operated so that protruding members 92 and 93 are pulled toward cutter main body 40 into a second configuration by operating wire 98. Protruding members 92 and 93 slide axially along bipolar cutter 18 through the interior thereof toward the base end side of bipolar cutter 18. Thus, for example, protruding members 92 and 93 move further toward the base-end side than electrodes 42 and 43 (tips 42a and 43a for tip members 92b and 93b), and protruding members 92 and 93 are contained within bipolar cutter 18 as shown in FIG. 25E. Electrodes 42 and 43 are thus exposed to perform a more effective treatment of site 86 as shown in FIG. 25F.

Length L is the axial distance between the distal edges 42a, 43a, of electrodes 42 and 43 and the distal ends 92b, 93b of protruding members 92 and 93. Either the electrodes or the protruding members can extend more distally than the other as the length L is adjusted as desired by gap adjusting mechanism 96. To treat bleeding site 86, length L is adjusted as shown in FIG. 25E, and electrodes 42 and 43 have been have been brought into contact with bleeding site 86. In this state, when bipolar cutter 18 is further advanced toward bleeding site 86, bleeding site 86 is guided toward electrodes 42 and 43, coming into contact with electrodes 42 and 43 as shown in FIG. 25F.

It is also possible for bipolar cutter 18 to advance simultaneously with the pulling of protruding members 92 and 93, and for electrodes 42 and 43 to come into contact with bleeding site 86, thereby skipping over the state shown in FIG. 25D.

When electrodes 42 and 43 are brought into contact with side branch 72 and bleeding site 86 and these events are confirmed by the surgeon, for example, the surgeon operates foot switch 80 of high frequency generating device 56, causing a high frequency current to flow. This results in the coagulation of the region where bleeding site 86, blood vessel connective tissue 7 1, or side branch 72 is in contact with body side electrode 42, and bleeding site 86 or side branch 72 is treated by cutting electrode 43. Accordingly, as shown in FIG. 26B, the portion in which blood vessel 61 was connected to blood vessel connective tissue 71 by side branch 72 is cut away by treating (cutting) side branch 72. At this time, body side electrode 42, with its large contact surface area, is positioned further to the top (body) side from blood vessel 61 than cutting electrode 43; hence, the effect of heat on blood vessel 61 is minimized. Further, bleeding site 86 is cauterized to stop the bleeding.

In this manner, due to the presence of gap 94, the object of examination is treated by simply pressing bipolar cutter 18 against it. That is, since it is not necessary to conduct any operation other than moving bipolar cutter 18 back and forth in treating the object of examination, the overall movement of the endoscopic tissue harvesting device as a whole is reduced, and operability is enhanced.

Further, in bipolar cutter 18, protruding members 92 and 93 are axially displaced along bipolar cutter 18 to the base end side thereof by gap adjusting mechanism 96, exposing electrodes 42 and 43. In this process, electrodes 42 and 43 are pressed against bleeding site 86. Thus, bipolar cutter 18 can readily treat bleeding site 86.

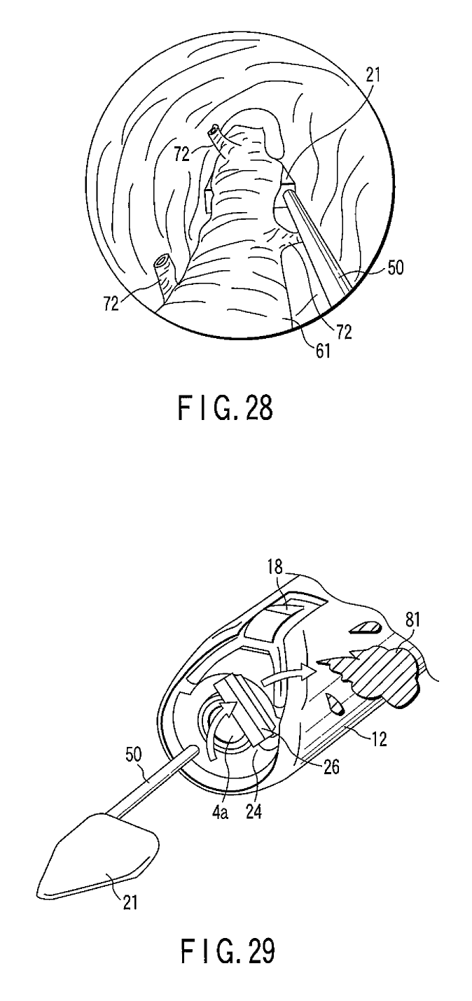

As set forth above, once side branch 72 or bleeding site 86 has been treated (as shown in FIG. 27), blood vessel holder 21 passes to the bottom of blood vessel 61, for example, lifting blood vessel 61. In this process, a determination is made as to whether side branch 72 is completely treated based on the monitor image shown in FIG. 28, for example, or whether bleeding site 86 has been completely treated based on a similar image, not shown.

Treating sheath 2 is pressed further into cavity interior 69, and cavity interior 69 is observed based on the monitor image. When blood vessel holder 21 approaches the next side branch 72, the same technique as that set forth above is repeated with bipolar cutter 18, side branch 72 is treated, and blood vessel 61 is cut away from blood vessel connective tissue 71.

When treating sheath 2 is pushed further into cavity interior 69 and protruding members 92 and 93 are pushed against wall 85, bleeding site 86 is cauterized to stop the bleeding in the same manner as above.

As this technique of treating side branch 72 and bleeding site 86 is repeated as set forth above, such adherent matter 81 blood, mucous, and subcutaneous fat 73 sometimes adhere to objective lens surface 4a, obstructing the visual field of rigid endoscope 4. In such cases, when the force exerted by torsion coil spring 29 is countered using a finger to rotate wiper operating member 28 while continuing to grip operating member cover 11, as shown in FIG. 29, wiper 24 rotates via wiper rod 25. Thus, scraping member 26a scrapes away the adherent matter 81 such as blood, mucous, and subcutaneous fat 73 that has adhered to objective lens surface 4a.

Wiper 24 is subject to the force of torsion coil spring 29. Thus, when the finger is removed from wiper operating member 28, wiper 24 recovers in the direction of withdrawal from objective lens surface 4a. Accordingly, by repeating the above-described operation several times, the stubborn adherent matter 81, such as subcutaneous fat 73, that is clinging to object lens surface 4a is cleanly scraped away. Further, since wiper 24 recovers in the direction of withdrawal from object lens surface 4a when the finger is removed from wiper operating member 28, the visual field of field of rigid endoscope 4 is not obstructed by wiper 24.

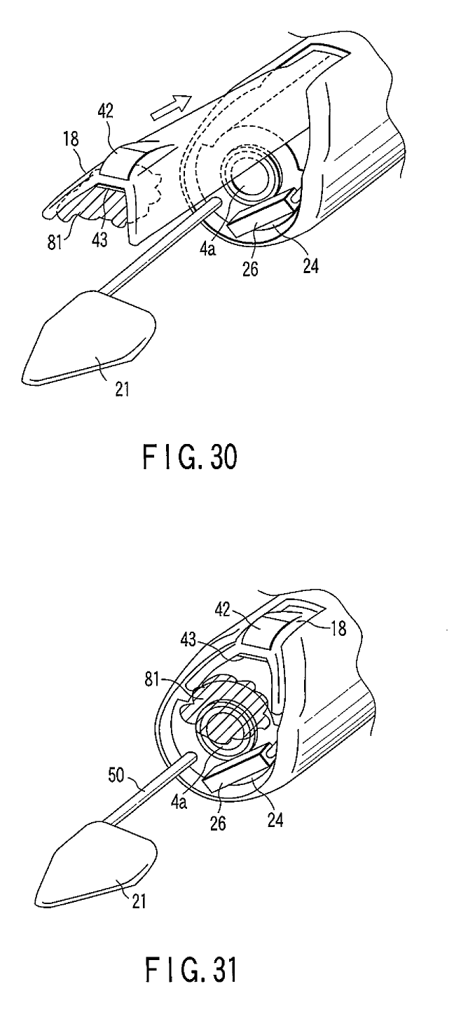

Further, when bipolar cutter 18 repeatedly treats (cuts) side branches 72 and bleeding sites 86, as shown in FIG. 30, adherent matter 81 such as mucous and subcutaneous fat 73 adheres to the inner surface of bipolar cutter 18 due to the roof-like shape of bipolar cutter 18 (the roof member covering cutter main body 40). However, when bipolar cutter 18 is withdrawn by cutter operation member 19 and is pulled into first treating apparatus channel 14, the mucous and subcutaneous fat 73 are scraped away by the front end surface of sheath main body 10. Accordingly, adherent matter 81 that has adhered to bipolar cutter 18 is readily scraped away. In the present embodiment, the clearance between bipolar cutter 18 and sheath main body 10 is set low in order to scrape away the mucous and subcutaneous fat 73 that have adhered to bipolar cutter 18 with the front end surface of sheath main body 10. This clearance to the space between the outer surface of bipolar cutter 18 and the inner surface of first treating apparatus channel 14.

Further, as shown in FIG. 31, the adherent matter 81 that has been wiped away sometimes adheres to objective lens surface 4a, obstructing the visual field. In this case as well, operating wiper operating member 28 as set forth above to rotate wiper 24 causes the adherent matter 81 adhering to object lens surface 4a to be scraped away.

The operation of wiping off adherent matter 81 adhered to bipolar cutter 18 and the operation of scraping off adherent matter 81 adhered to objective lens surface 4a are repeated. The task of treating (cutting) side branch 72 to separate blood vessel 61 from blood vessel connective tissue 71 and the task of treating bleeding sites 86 are repeated. When this process advances to the inguinal region, the treating (cutting) of side branches 72 and bleeding sites 86 is halted. A scalpel or the like is then used to form an incision in inguinal region 63 directly above blood vessel 61. Blood vessel 61 is pulled out through the incision and the two cut ends of blood vessel 61 are then ligated with sutures.

Next, blood vessel 61 is removed from incision 64 toward the ankle, eventually yielding a single blood vessel of about 60 cm. The technique is basically identical to that employed to obtain blood vessel 61 from knee 62 to inguinal region 63 as set forth above, and the description thereof is omitted.

In this manner, in the present embodiment, gap adjusting mechanism 96 can be employed in the course of bringing protruding members 92 and 93 into contact with wall 85 to displace protruding members 92 and 93 further to the base end side of bipolar cutter 18 than electrodes 42 and 43, for example, readily exposing electrodes 42 and 43. Thus, in the present embodiment, exposed electrodes 42 and 43 can be pressed against bleeding site 86, for example, permitting the ready treatment of bleeding site 86.

That is, in the present embodiment, a bleeding site 86, for example, can be guided to electrodes 42 and 43 by guiding member 91, permitting the ready treatment of bleeding site 86, by using gap adjusting mechanism 96 to adjust length L as desired so that protruding members 92 and 93 are about evenly aligned with electrodes 42 and 43, or are positioned somewhat further toward the base end side than electrodes 42 and 43.

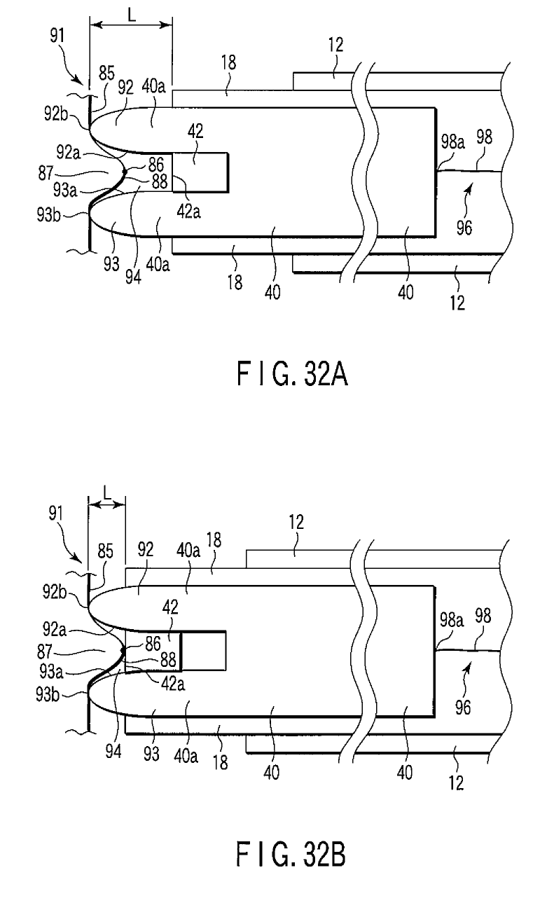

Although protruding members 92 and 93 are contained within bipolar cutter 18 and positioned further toward the base end side than electrodes 42 and 43 to expose electrodes 42 and 43 for the treatment of bleeding site 86, for example, in the present embodiment, the mode of implementation is not limited thereto. For example, as shown in FIG. 32A, when there is a convex portion 87 in wall 85 and a bleeding site 86 is located at the tip 88 of convex portion 87, convex portion 87 is sandwiched between protruding members 92 and 92 (the convex portion is positioned within gap 94), with bleeding site 86 facing electrodes 42 and 43. In this process, as shown in FIG. 32B, length L is narrowed by means of gap adjusting mechanism 96, protruding members 92 and 93 are positioned further forward than electrodes 42 and 43, and electrodes 42 and 43 are brought into contact with bleeding site 86 in an unexposed state and treat electrodes 42 and 43.

Portions of the drawings, such as lead wire 44 and insulating coating 46, have been omitted in FIGS. 32A and 32B.

As such, in the present embodiment, length L is adjusted as desired with gap adjusting mechanism 96 based on the size of bleeding site 86, rendering possible the treatment of bleeding site 86 even when protruding members 92 and 93 are positioned further forward than electrodes 42 and 43. That is, in the present embodiment, there is no need to exposed electrodes 42 and 43 in the course of treating bleeding site 86 by displacing protruding members 92 and 93 so that they are roughly even with electrodes 42 and 43, or somewhat further toward the base end side of bipolar cutter 18 than electrodes 42 and 43. In other words, the second configuration of the guiding member and treating electrode is obtained my activating the gap adjusting mechanism to a position between the maximum and minimum positions.

Further, in the present embodiment, adjusting length L as desired by positioning protruding members 92 and 93 further forward than electrodes 42 and 43 with gap adjusting mechanism 96 permits the guiding of blood vessel 61, for example, to electrodes 42 and 43 with guiding member 91, permitting the ready treatment of blood vessel 61.

In the present embodiment, adjusting length L as desired with gap adjusting mechanism 96 and guiding the object of examination to electrodes 42 and 43 with guiding member 91 in this manner makes it possible to readily bring an object of examination, such as a bleeding site 86 or a side branch 72 into contact with electrodes 42 and 43, permitting ready treatment of the object of examination.

In the present embodiment, it is possible to treat blood vessel 61, bleeding site 86, and the like without conducting further operations, such as pressing down on and displacing a blade or opening and closing a row. In the present embodiment, since treating is possible by means of simple back-and-forth movement without further operations, few operational errors occur. Accordingly, the treatment errors due to operational errors can be prevented.

An example of variation of Embodiment 1 will be described next. Items identical to those in above-described Embodiment 1 are denoted by the same numbers and the detailed description thereof is omitted.

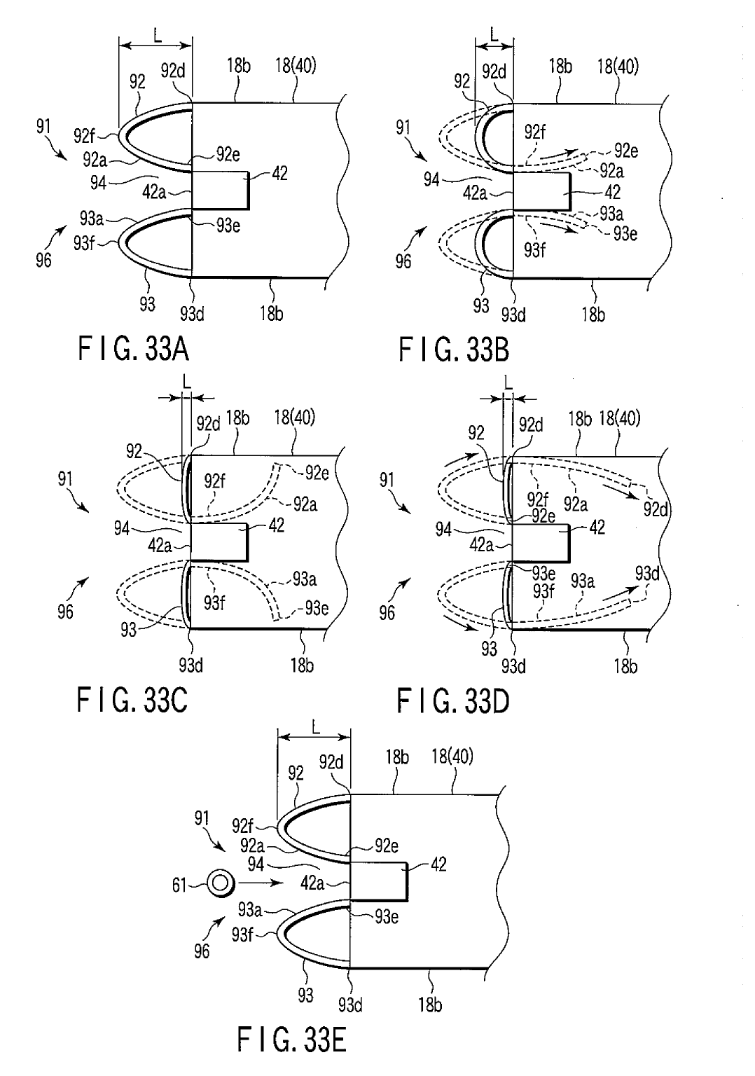

A first variation example will be described with reference to FIGS. 33A, 33B, 33C, 33D, and 33E. In these figures, portions such as lead wire 44 and insulation coating 46 have been omitted. In this variation, guiding member 91 inherently performs as the gap adjusting mechanism 96 as a result of forces applied by the patient's tissue against guiding member 91. Protruding members 92 and 93 are made, for example, of a wire-like shape memory alloy of pointed shape. As shown in FIG. 33A, one end 92d and 93d of each of protruding members 92 and 93 is secured to the outer circumference 18b of bipolar cutter 18, for example, and the other end 92e and 93e is slidably positioned within bipolar cutter 18 in the vicinity of electrodes 42 and 43 in such a manner as to not exit bipolar cutter 18. Middle portions 92f and 93f are positioned to the fore of electrodes 42 and 43. Length L is adjusted as desired by sliding ends 92e and 93e.

In the present variation example, protruding members 92 and 93 are not limited to bipolar cutter 18, and can be secured to the outer circumference of cylindrically shaped cutter main body 40, for example.

Protruding members 92 and 93 cause ends 92e and 93e to slide by coming into contact with a wall 85, not shown, thereby producing the variation shown in FIG. 33B, wherein guiding member 91 (protruding members 92 and 93), doubling as gap adjusting mechanism 96, adjusts length L by the appropriate amount. More specifically, for example, protruding members 92 and 93, as shown in FIG. 33B, have one end 92d and 93d that serves as the base point, and are contained within bipolar cutter 18 by sliding the other end 92e and 93e. Middle sections 92f and 93f are aligned roughly evenly with electrodes 42 and 43, positioned further toward the base end side 25 than electrodes 42 and 43, or are positioned further forward than electrodes 42 and 43.

Thus, as in Embodiment 1, for example, electrodes 42 and 43 are positioned roughly evenly with guiding member 91 as shown in FIG. 33C or forward of the guiding member, and are exposed from guiding member 91.

As shown in FIG. 33D, protruding members 92 and 93 may be positioned containably within bipolar cutter 18 in such a manner that the other ends 92e and 93e are secured in the vicinity of electrodes 42 and 43 of bipolar cutter 18, for example, and ends 92d and 93d are slidable along the inner circumferential surface 18c of bipolar cutter 18. Thus, by bringing protruding members 92 and 93 into contact with wall 85 (not shown), middle sections 92f and 93f are aligned roughly evenly with electrodes 42 and 43, positioned somewhat toward the base end side than electrodes 42 and 43, or positioned forward of electrodes 42 and 43.

Protruding members 92 and 93 are made of a shape memory alloy. Thus, they deform as shown in FIGS. 33B, 33C, and 33D. After moving back from wall 85, protruding members 92 and 93 contained in bipolar cutter 18 protrude from bipolar cutter 18, and their tips assume positions forward of electrodes 42 and 43, returning to the state shown in FIG. 33A.

As a matter of course, protruding members 92 and 93, as shown in FIG. 33E, can guide blood vessel 61 to electrodes 42 and 43 in the same manner as in Embodiment 1 without deforming.

As shown in FIGS. 32A and 32B, protruding members 92 and 93 can be positioned forward of electrodes 42 and 43 so that electrodes 42 and 43 are not exposed.

In the present variation example, length L can be adjusted by simply doubling guiding member 91 (protruding members 92 and 93) as gap adjusting mechanism 96 and bringing protruding members 92 and 93 into contact with wall 85; and, for example, protruding members 92 and 93 can be contained in bipolar cutter 18. Thus, the present variation example makes it possible to readily guide bleeding site 86 to electrodes 42 and 43 by a simple operation, and bleeding site 86 can be easily pressed against electrodes 42 and 43 and treated.

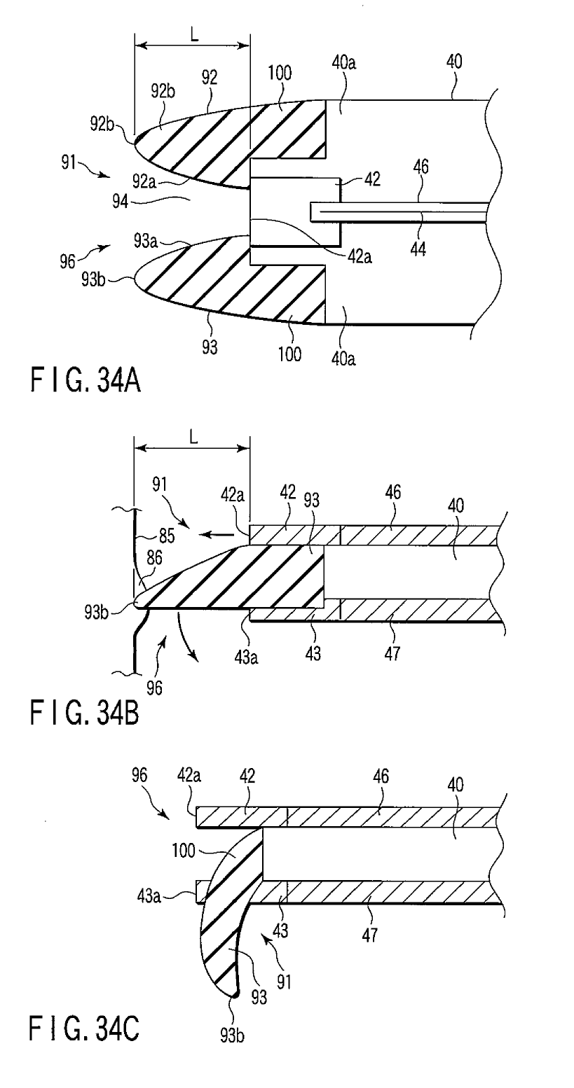

In another variation shown in FIG. 34A, treating tip member 40a provided on the tip of cutter main body 40, protruding members 92 and 93 can be formed of a soft material (such as an elastic member 100 or resin). Thus, in the course of bringing protruding members 92 and 93 into contact with wall 85, they distort readily as shown in FIGS. 34B and 34C. Thus, the above-described results can be achieved and costs can be reduced in the present variation example.

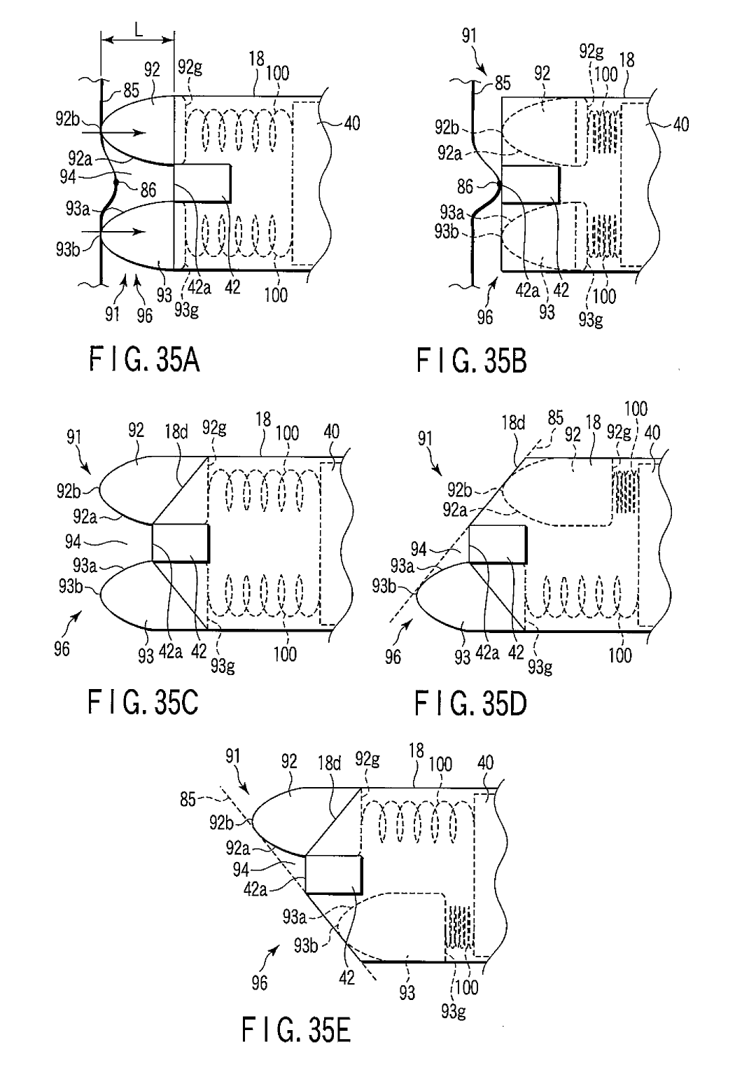

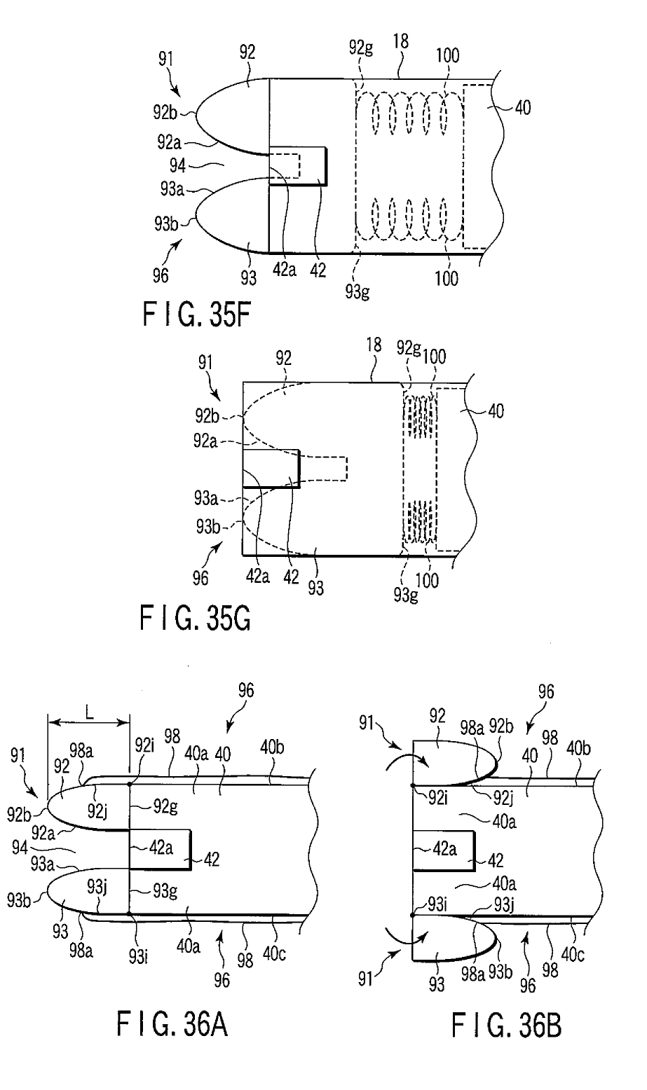

Another variation example will be described with reference to FIGS. 35A and 35B. In these figures, such portions as lead wire 44 and insulation coating 46 have been omitted. Guiding member 91 (protruding members 92 and 93) doubles as gap adjusting mechanism 96 in the same manner as in the first variation example. As shown in FIG. 35A, elastic members 100, such as springs, that adjust gap length L as desired by displacing protruding members 92 and 93 in the longitudinal direction of cutter main body 40 by exerting forces on protruding members 92 and 93 in the longitudinal direction of cutter main body 40 are present on base end members 92g and 93g. Elastic members 100 desirably exert a force (elastic force) causing protruding members 92 and 93 to be pushed further forward than electrodes 42 and 43. Protruding members 92 and 93 are linked to cutter main body 40 through elastic members 100.

In the present variation example, when protruding members 92 and 93 are brought into contact with wall 85 by pressure (a contact force) greater than or equal to the elastic force, as shown in FIG. 35A, elastic members 100 compress and protruding members 92 and 93 move in the axial direction of bipolar cutter 18. This adjusts gap length L as desired. In this process, protruding members 92 and 93 can be contained in bipolar cutter 18, for example.

Thus, in the same manner as in Embodiment 1, electrodes 42 and 43 are aligned roughly evenly with guiding member 91, or are positioned forward of guiding member 91, causing them to be exposed on guiding member 91, as shown in FIG. 35B. As shown in FIGS. 32A and 32B, protruding members 92 and 93 can also be positioned forward of electrodes 42 and 43, so that electrodes 42 and 43 are not in an exposed state.

By separating from wall 85, for example, protruding members 92 and 93 are subjected to the elastic force of flexible members 100, and protrude from bipolar cutter cutter 18. Thus, as shown in FIG. 35A, protruding members 92 and 93 return to a state where they are positioned forward of electrodes 42 and 43.

Hence the same effect can be obtained from the present variation example as in the first variation example. Further, in the present variation example, protruding members 92 and 93 can be rapidly returned to their original state (the state in which tips 92a and 93a are positioned forward of tips 42a and 42b) by elastic member 100 upon separation from wall 85. Thus, in the present variation example, for example, another bleeding site 86 can be rapidly guided to electrodes 42 and 43, and bleeding site 86 can be readily pressed against electrodes 42 and 43 and treated.

In the present variation example, as shown in FIG. 35C, bipolar cutter 18 can be in the shape of a truncated cone and protruding members 92 and 93 can protrude from inclined surface 18d of cutter main body 40. Thus, in the present variation example, as shown in FIGS. 35D and 35E, in the course of causing bipolar cutter 18 to move at an angle relative to wall 85, just the protruding member that approaches wall 85 and comes into contact with wall 85 (protruding member 92 in FIG. 35D and protruding member 93 in FIG. 35E) will be displaced in the axial direction of tip cover 12 by an elastic member 100, and electrodes 42 and 43 can be exposed. In this manner, in the present variation example, the above-described effects can be achieved and operating efficiency can be promoted even when bipolar cutter 18 is caused to move at an angle relative to wall 85.

In the present variation example, protruding members 92 and 93 can be mutually connected to base end members 92g and 93g and integrated within bipolar cutter 18, as shown in FIGS. 35F and 35G.

Embodiment 2 will be described with reference to FIGS. 36A and 36B. Items identical to those in above-described Embodiment 1 are denoted by the same numbers and the detailed description thereof is omitted. Portions have been omitted from FIGS. 36A and 36B.

In Embodiment 2, gap adjusting mechanism 96 adjusts gap length L as desired by causing protruding members 92 and 93 to rotate about reference points 92i and 93i thereof toward lateral surfaces 40b and 40c of cutter main body 40. In the present embodiment, operation wire 98 pushes and pulls protruding members 92 and 93, causing protruding members 92 and 93 to rotate to lateral surfaces 40b and 40c, thereby adjusting length L as desired.

In the present embodiment, protruding members 92 and 93 rotate (away from electrodes 42 and 43), when pulled by operating wire 98, toward the lateral surface of cutter main body 40 with reference to a plane roughly even with electrodes 42 and 43.

In the present embodiment, protruding member 92 rotates about reference point 92i, moving to the right lateral surface 40b side of cutter main body 40. Reference point 92i is the area of contact between base end member 92g of protruding member 92 and the tip of right lateral surface 40b.

In the present embodiment, protruding member 93 rotates about reference point 93i, moving to the left lateral surface 40c side of cutter main body 40. Reference point 93i is the area of contact between base end members 93g of protruding member 93 and left lateral surface 40c of cutter main body 40.

Reference points 92i and 93i are rotational axes permitting rotation in a circumferential direction orthogonal to the axial direction of cutter main body 40 (bipolar cutter 18) and the direction of a straight line connecting protruding members 92 and 93.

Protruding members 92 and 93 are disposed symmetrically about electrodes 42 and 43.

On tip 98a, lateral surface 92j on the reference point 92i side of protruding member 92 connects with lateral surface 93j of the reference point 93i side of protruding member 93. The other end of operating wire 98 is inserted into bipolar cutter 18 and connected to cutter operating member 19.

When cutter operating member 19 is operated, protruding members 92 and 93 are pulled by operating wire 98. In this process, protruding member 92 rotates to the right lateral surface 40b side about reference point 92i, as shown in FIG. 36B, and protruding member 93 rotates to the left lateral surface 40c side about reference points 93i, as shown in FIG. 36B.

Thus, in the present embodiment, the same effects can be achieved as in Embodiment 1.

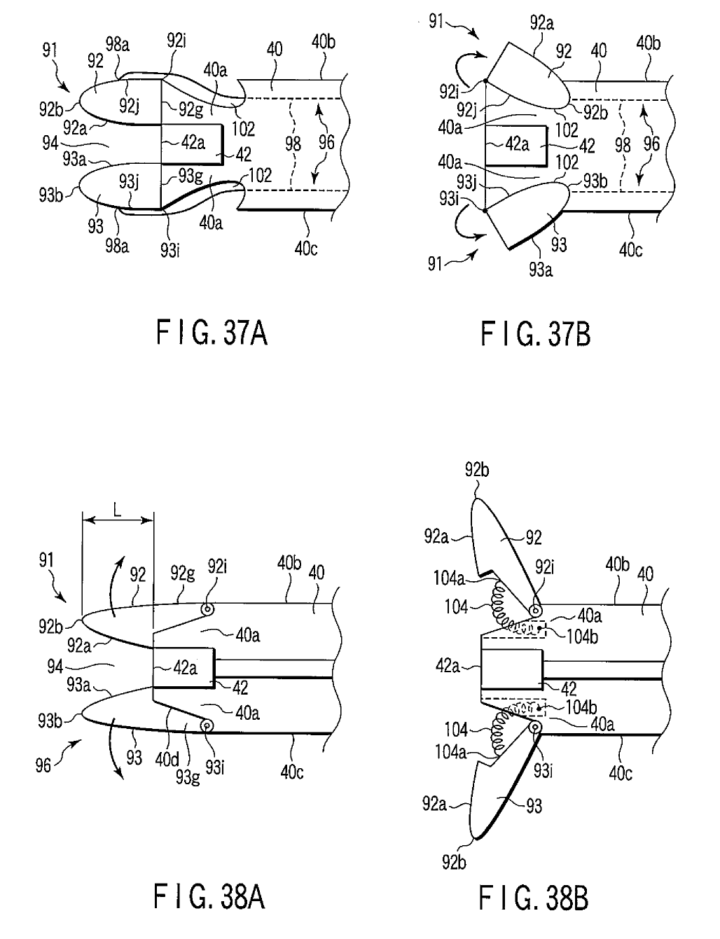

A first variation example of the present embodiment will be described. As shown in FIG. 37A, in cutter main body 40, housing members 102 housing rotating protruding members 92 and 93 can be present on right lateral surface 40b and left lateral surface 40c. Thus, protruding members 92 and 93 are rotated by a larger angle than that described above when pulled by operating wire 98. Thus, in the course of rotating protruding members 92 and 93 and housing them within housing member 102, the diameter of cutter main body 40 containing electrodes 42 and 43 and protruding members 92 and 93 is reduced. Thus, the present variation example promotes operating efficiency within body cavities.

Operating wire 98 is inserted into cutter main body 40 through housing member 102, and is connected to cutter operating member 19.

Portions such as lead wire 44 and insulation coating 46 are omitted in the various figures set forth above.

A second variation example will be described next. Guiding member 91 (protruding members 92 and 93) doubles as gap adjusting mechanism 96. As shown in FIG. 38A, cutter main body 40 has the shape of a truncated cone in the same manner as in the bipolar cutter 18 shown in FIG. 35C. In the course of being brought into contact with wall 85, protruding members 92 and 93 can be rotated about reference points 92i and 93i toward the lateral surface of cutter main body 40 in a manner separating them from inclined surface 40d.

In the present Embodiment, reference points 92i and 93i are areas of contact between base end members 92g and 93g of protruding members 92 and 93, and the furthest base end side (the frontmost side of right lateral surface 40b and the frontmost side of left lateral surface 40c) of inclined surface 40d.

An elastic member 104 is present in the form of a material exerting an energizing force when rotating protruding members 92 and 93 are in the closed state shown in FIG. 38A where the longitudinal direction of rotating protruding members 92 and 93 is roughly parallel to the longitudinal direction of cutter main body 40 in the course of rotating protruding members 92 and 93 toward lateral surfaces 40b and 40c of cutter main body 40 about reference points 92i and 93i. More specifically, elastic member 104 is, for example, a spring exerting an elastic force on protruding members 92 and 93 that have been rotated toward right lateral surface 40b and left lateral surface 40c, causing them to rotate about reference points 92i and 93i to the inside of cutter main body 40. One end 104a of elastic member 104 is secured to the end surface of protruding members 92 and 93, and the other end 104b is secured within cutter main body 40.

When protruding members 92 and 93 have been brought into contact with wall 85 (not shown in FIG. 38A) by a pressure (contact force) greater than or equal to the elastic force of elastic member 104, protruding members 92 and 93 rotate about reference points 92i and 93i toward right lateral surface 40b and left lateral surface 40c as shown in FIG. 38B.

Thus, in the same manner as in Embodiment 1, electrodes 42 and 43 are aligned roughly evenly with guiding member 91 as shown in FIG. 38B, or are positioned forward of guiding member 91, and are exposed from guiding member 41.

When protruding members 92 and 93 are moved away from wall 85, the elastic force of elastic member 100 causes protruding members 92 and 93 to rotate about reference points 92i and 93i toward the inside of cutter main body 40, returning to the state shown in FIG. 38A.

Thus, in the present variation example, combining guiding member 91 (protruding members 92 and 93) and gap adjusting mechanism 96 and bringing protruding members 92 and 93 into contact with wall 85 rotates protruding members 92 and 93, permitting adjustment of length L. Thus, in the present variation example, bleeding site 86 can be readily guided to electrodes 42 and 43 by an easy operation, permitting bleeding site 86 to be readily pressed against electrodes 42 and 43 and treated.

In the present variation example, elastic member 100 allows protruding members 92 and 93 to rapidly return to their original states upon separation thereof from wall 85. Thus, in the present variation example, another bleeding site 86, for example, can be quickly guided to electrodes 42 and 43, making it possible to readily bring bleeding site 86 into contact with electrodes 42 and 43 for treatment.

In this manner, roughly the same effects can be achieved in the present variation example as in the first and second variation examples of Embodiment 1.

In the present variation example, protruding members 92 and 93 can also be pulled by an operating wire 98, not shown.

Further, in the present variation example, in the same manner as in the second variation example of Embodiment 1 shown in FIGS. 35D and 35E, it is possible to rotate just the protruding member 92 or 93 that comes into contact with wall 85, exposing electrodes 42 and 43, in the course of causing bipolar cutter 18 (cutter main body 40) to move at an angle. Thus, in the present variation example, the same effect can be achieved as in the second variation example of Embodiment 1 shown in FIGS. 35D and 35E.

Portions such as lead wire 44 and insulation coating 46 have been omitted in the above figures.

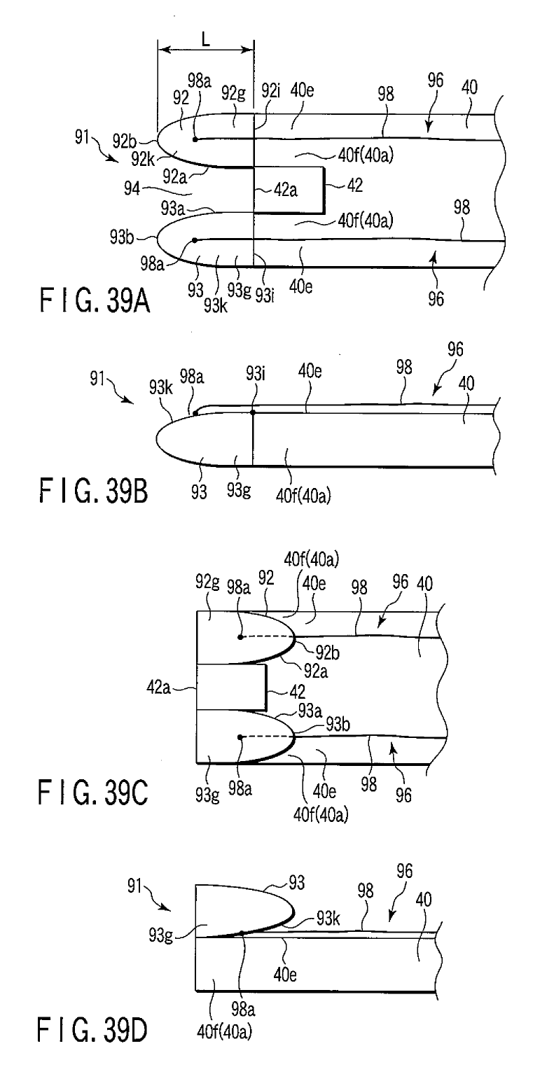

Embodiment 3 will be described next with reference to FIGS. 39A, 39B, 39C, and 39D. Items identical to those in above described Embodiment 1 have been identically numbered and the detailed description thereof has been omitted. Portions have been omitted in FIGS. 39A, 39B, 39C, and 39D.

The gap adjusting mechanism adjusts the gap as desired by rotating the protruding members toward the upper surface side of the main body.

In the present embodiment, gap adjusting mechanism 96 rotates protruding members 92 and 93 toward the upper surface 40e side of cutter main body about base end members 92i and 93i of protruding members 92 and 93, thereby adjusting length L as desired. Operating wire 98 in the present embodiment pushes and pulls protruding members 92 and 93, causing them to rotate to the upper surface 40e side, thereby adjusting gap L as desired.

In the present embodiment, protruding members 92 and 93 are pulled by operating wire 98 to rotate them toward the upper surface of cutter main body 40, so that they are roughly even with electrodes 42 and 43.

In the present embodiment, protruding member 92 rotates about reference point 92i, moving to the upper surface 40e side of cutter main body 40. Reference point 92i is the area of contact between base end member 92g of protruding member 92 and the frontmost end member 40f (treating tip member 40a) of cutter main body 40.

In the present embodiment, protruding member 93 rotates about reference point 93i, moving to the upper surface 40e side. Reference point 93i is the area of contact between base end member 93g of protruding member 93 and the frontmost end member 40f (treating tip member 40a) of cutter main body 40.

Further, in the present embodiment, reference points 92i and 93i are axes of rotation permitting rotation in a circumferential direction along a line connecting protruding members 92 and 93.

At tip 98a, for example, upper surface 92k of protruding member 92 and upper surface 93k of protruding member 93 are connected. The other end of operating wire 98 is inserted into bipolar cutter 18 and connects with cutter operating member 19.

When cutter operating member 19 is operated, protruding members 92 and 93 are pulled by operating wire 98. In this process, protruding member 92 rotates to the upper surface 92k side about reference points 92i, as shown in FIG. 39C, and protruding member 93 rotates to the upper surface 93k side about reference point 93i, as shown in FIGS. 39C and 39D.

Thus, the present Embodiment achieves the same effects as Embodiment 1.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscopic surgical device comprising:
a main body for extending into a surgical cavity created within a patient's body;
a treating electrode deployable within the surgical cavity from a distal end of the main body to sever target tissue of the patient's body; and
a guiding member for guiding target tissue to the treating electrode as the treating electrode advances within the surgical cavity;
wherein the guiding member and the treating electrode are movable with respect to each other between a first configuration wherein the guiding member extends distally beyond the treating electrode and a second configuration wherein the extension of the guiding member distally beyond the treating electrode is less than the first configuration, wherein the first configuration is adapted to treat target tissue comprised of narrow tissues in the patient's body, and wherein the second configuration is adapted to treat target tissue comprising substantially flat tissue surfaces of the surgical cavity.

2. The device of claim 1 wherein the treating electrode is slidable along the guiding member.

3. The device of claim 1 wherein the treating electrode extends distally beyond the guiding member when in the second configuration.

4. The device of claim 1 further comprising:
a handle coupled to a proximal end of the main body; and
a manually-operable actuator on the handle and coupled to the guiding member through the main body for moving the guiding member between the first and second configurations.

5. The device of claim 1 wherein the guiding member is comprised of parallel protruding members oriented in the first configuration to form a gap that extends longitudinally, the gap having an open distal end, side edges formed by the protruding members, and a proximal edge formed by the treating electrode.

6. The device of claim 5 wherein the parallel protruding members are tapered at their distal ends for guiding the target tissue toward the treating electrode.

7. The device of claim 5 wherein the protruding members comprise plates of shape-memory material.

8. The device of claim 5 wherein the protruding members comprise wires of shape-memory material.

9. The device of claim 5 wherein the gap has a longitudinal length from the treating electrode to the open distal end that is varied between the first and second configurations, and wherein the device further comprises a gap adjusting mechanism for manually controlling the longitudinal length of the gap.

10. The device of claim 9 wherein the gap adjusting mechanism adjusts the longitudinal length as desired by causing the protruding members to be displaced in the longitudinal direction from the main body.

11. The device of claim 10 wherein the gap adjusting mechanism comprises:
an operating member outside the surgical cavity that is manually movable by a user; and
a connecting wire coupled between the protruding members and the operating member for transmitting motion of the operating member to the protruding members to change the longitudinal length of the gap.

12. The device of claim 11 wherein the protruding members comprise formed wires of shape-memory material, and wherein the formed wires are an extension of the connecting wire.

13. The device of claim 12 wherein the formed wires slide in the proximal direction to obtain the second configuration.

14. The device of claim 5 wherein the protruding members are comprised of a flexible material, and wherein the protruding members are moved into the second configuration by being deflected when contacting the patient's body.

15. The device of claim 5 further comprising elastic members, wherein each elastic member mounts a respective protruding member to the main body, and wherein the protruding members are moved into the second configuration by compressing at least one elastic member when contacting the patient's body.

16. The device of claim 5 wherein the protruding members are pivotally mounted to the main body at their proximal ends, and wherein the protruding members rotate between the first and second configurations.

17. The device of claim 16 further comprising an adjusting mechanism for manually controlling the configuration of the protruding members, wherein the adjusting mechanism comprises:
an operating member outside the surgical cavity that is manually movable by a user; and
a connecting wire coupled between the protruding members and the operating member for transmitting motion of the operating member to the protruding members to rotate the protruding members between the first and second configurations.
